(19) United States
(12) Patent Application Publication
(10) Pub. No.: US 2022/0259195 A1
(43) Pub. Date: Aug. 18, 2022

(54) CRYSTALLINE FORMS OF A CYCLIN-DEPENDENT KINASE INHIBITOR

(71) Applicant: Nuvation Bio Inc., New York, NY (US)

(72) Inventors: Matthew John BIGERT, Allentown, PA (US); Michael F. BRADLEY, Rahway, NJ (US); Thomas Stearns TEMPLEMAN, Chesterfield, MO (US); Robert M. WENSLOW, Cream Ridge, NJ (US)

(21) Appl. No.: 17/591,186

(22) Filed: Feb. 2, 2022

Related U.S. Application Data
(60) Provisional application No. 63/145,362, filed on Feb. 3, 2021.

(51) Int. Cl. C07D 413/14; A61K 45/06; A61K 31/538; A61P 35/00

(52) U.S. Cl. CPC C07D 413/14; A61K 45/06; C07B 2200/13; A61P 35/00; A61K 31/538

(57) ABSTRACT

Provided herein are crystalline forms of a cyclin-dependent kinases (CDK) inhibitor, compositions thereof, methods of preparation thereof, and methods of use thereof.

### CROSS-REFERENCE TO RELATED APPLICATIONS

This application claims priority to U.S. Provisional Application No. 63/145,362, filed Feb. 3, 2021, the contents of which are incorporated herein by reference in their entirety.

### FIELD

Provided herein are crystalline forms of a cyclin-dependent kinases (CDK) inhibitor, compositions thereof, methods of preparation thereof, and methods of use thereof.

### BACKGROUND

The cell cycle is a period between the successive divisions of a cell. During this period, the contents of the cell must be accurately replicated. The processes that permit the cell to divide are very precisely controlled by a multitude of enzymatic reactions amongst which the protein kinase-triggered protein phosphorylation plays a major role. In eukaryotes, there are four main stages/phases of cell cycle namely the Gap-1 (G1) phase, Synthesis (S) phase, Gap-2 (G2) and Mitosis (M) phases. An extended phase of Gap-1 phase is coined as Gap-0 (G0) phase or Resting phase (Cancers 2014, 6, 2224-2242).

Uncontrolled proliferation is the hallmark of cancer and other proliferative disorders and abnormal cell cycle regulation is, therefore, common in these diseases. Cyclin-dependent kinases (CDK) constitute a heterodimeric family of serine/threonine protein kinases involved in cell cycle and transcription. They include two main groups: cell cycle CDK and transcriptional CDK. The functionality of CDK depends on specific interactions with regulatory proteins named cyclins which form heterodimeric complexes with their partners. These complexes are important regulators of the cellular processes, especially in the cell cycle progression.

The human proteome contains 20 CDK along with 29 cyclins. CDK1, CDK2, CDK4 and CDK6 are generally considered cell cycle CDK, whereas CDK7, CDK8, CDK9 and CDK11 are mainly involved in transcription regulation (Genome Biol 2014; 15 (6):122, Nat Cell Biol 2009; 11 (11):1275-6). CDKS is the prototype of atypical CDK: it is activated by the non-cyclin proteins p35 (or Cdk5R1) and p39 (or Cdk5R2) and has unique post-mitotic functions in neuronal biology, angiogenesis and cell differentiation. Proliferative signals induce the transition from the G0 or G1 phases into S phase through the activation of the structurally related CDK4 and CDK6 [Development, 2013; 140 (15):3079-93, Biochem Pharmacol 2012; 84 (8):985-93, Nature 2014; 510 (7505):393-6]. The binding of cyclin D to CDK4 and to CDK6 promotes the phosphorylation of the transcriptional repressor retinoblastoma protein (RB1).

CDK hyperactivity is often observed in cancer, reflecting their prominent role in cell cycle and transcription regulation. In cancer cells, the process of cell division becomes unregulated, resulting in uncontrolled growth that leads to the development of a tumor. A number of mechanisms contribute to the dysregulation of the cell cycle in malignant cells, including the amplification and hyperactivity of CDK4/6, or their genomic instability, which might cause CDK4/6 to become oncogenic drivers of cell replication. Usurping these mechanisms, cancer cells can continue to replicate by triggering the G1 to S phase transition. This process appears to be facilitated by a shortening of the G1 phase. In a cancer cell, CDK4/6 antagonizes intrinsic tumor suppression mechanisms including cell senescence and apoptosis, which further augments the growth of a tumor. Cancer cells also upregulate other CDK and cyclins and decrease suppressive mechanisms such as intrinsic CDK inhibitors and tumor suppressor proteins. The overall effect of this type of cell cycle dysregulation is malignant cell proliferation and the development of cancer (Clinical Breast Cancer, 2016, 1526-8209).

The development of therapies, including monotherapies, for treatment of proliferative disorders using a therapeutic targeted generically at CDK, or specifically at dual inhibition of CDK4 and CDK6, is therefore potentially highly desirable. U.S. Patent Publication No. US 2019/0248774 A1 discloses 5-fluoro-4-(8-fluoro-4-isopropyl-3,4-dihydro-2H-benzo[b][1,4]oxazin-6-yl)-N-(5-(1-methylpiperidin-4-yl)pyridin-2-yl)pyrimidin-2-amine (hereinafter “Compound F”) having the structure shown below, which is a potent CDK4/6 inhibitor.

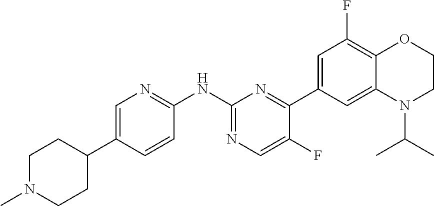 [SMILES: CC(C)N1CCOc2c(F)cc(-c3nc(Nc4ccc(C5CCN(C)CC5)cn4)ncc3F)cc21]

Crystalline forms of Compound 1 are disclosed herein. The crystalline forms disclosed herein may provide the advantages of bioavailability and stability and may be suitable for use as an active agent in a pharmaceutical composition. Variations in the crystal structure of a pharmaceutical drug substance may affect the dissolution rate (which may affect bioavailability, etc.), manufacturability (e.g., ease of handling, ease of purification, ability to consistently prepare doses of known strength, etc.) and stability (e.g., thermal stability, shelf life (including resistance to degradation), etc.) of a pharmaceutical drug product. Such variations may affect the methods of preparation or formulation of pharmaceutical compositions in different dosage or delivery forms, such as solid oral dosage forms including tablets and capsules. Compared to other forms such as non-crystalline or amorphous forms, crystalline forms may provide desired or suitable hygroscopicity, particle size control, dissolution rate, solubility, purity, physical and chemical stability, manufacturability, yield, reproducibility, and/or process control. Thus, the crystalline forms disclosed herein may provide advantages of improving the manufacturing process of an active agent or the stability or storability of a drug product form of the active agent, or having suitable bioavailability and/or stability as an active agent.

### BRIEF SUMMARY

In one aspect, provided herein is a crystalline form of Compound 1, as disclosed herein.

In another aspect, provided herein is a method of preparing a crystalline form of Compound 1, as disclosed herein.

In another aspect, provided herein is a composition, comprising a crystalline form of Compound 1, such as a pharmaceutical composition comprising a crystalline form of Compound 1, as disclosed herein.

In another aspect, provided herein is a kit comprising a crystalline form of Compound 1, as disclosed herein.

In another aspect, provided is a method of treating a proliferative disorder such as cancer in an individual in need thereof, comprising administering to the individual a therapeutically effective amount of a crystalline form of Compound 1, as detailed herein. Also provided is a method of modulating CDK1/and/or CDK2 and/or CDK4 and/or CDK6 in an individual, comprising administering to the individual a crystalline form of Compound 1, as detailed herein. Also provided is a crystalline form of Compound 1, as detailed herein, for use in therapy. Also provided is a crystalline form of Compound 1, as detailed herein, for use in a method of treating a proliferative disorder such as cancer. Also provided is use of a crystalline form of Compound 1, as detailed herein, in the manufacture of a medicament for treating a proliferative disorder such as cancer.

### BRIEF DESCRIPTION OF THE DRAWINGS

FIG. 1A shows an X-ray powder diffraction (XRPD) pattern of a substantially anhydrous crystalline form of Compound 1 (Form I).

FIG. 1B shows differential scanning calorimetry (DSC) and thermogravimetric analysis (TGA) graphs of Form I.

FIG. 1C shows a dynamic vapor sorption (DVS) graph of Form I.

FIG. 2A shows an XRPD patterns of a crystalline form of a chloroform solvate of Compound 1 (Form II).

FIG. 2B shows DSC and TGA graphs of Form II.

FIG. 3A shows an XRPD pattern of a crystalline form of Compound 1 (Form III).

FIG. 3B shows DSC and TGA graphs of Form III.

FIG. 4A shows an XRPD pattern of a crystalline form of a hydrate of Compound 1 (Form IV).

FIG. 4B shows DSC and TGA graphs of Form IV.

FIG. 5 shows a comparison between XRPD patterns of Form I and XRPD patterns of Form V.

### DETAILED DESCRIPTION

### Definitions

As used herein, unless clearly indicated otherwise, use of the terms “a”, “an” and the like refers to one or more.

As used herein, reference to “about” a value or parameter herein includes (and describes) embodiments that are directed to that value or parameter per se. For example, description referring to “about X” includes description of “X”. As used herein, and unless otherwise specified, the terms “about” and “approximately,” when used in connection with doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in this context, contemplate a dose, amount, or weight percent within 20%, within 15%, within 10%, within 5%, within 4%, within 3%, within 2%, within 1%, or within 0.5% of the specified dose, amount, or weight percent. Similarly, the terms “about” and “approximately,” when used in connection with a numeric value or range of values, indicate that the numeric value or range of values may vary within 20%, within 15%, within 10%, within 5%, within 4%, within 3%, within 2%, within 1%, or within 0.5% of the specified value or range.

As used herein, the term “crystalline form” refers to a crystalline solid form of a chemical compound, including, but not limited to, a single-component or multiple-component crystal form, e.g., a polymorph of a compound; or a solvate, a hydrate, a clathrate, a cocrystal, a salt of a compound, or a polymorph thereof. The term “crystal forms” and related terms herein refers to the various crystalline modifications of a given substance, including, but not limited to, polymorphs, solvates, hydrates, co-crystals and other molecular complexes, as well as salts, solvates of salts, hydrates of salts, other molecular complexes of salts, and polymorphs thereof. Crystal forms of a substance can be obtained by a number of methods, as known in the art. Such methods include, but are not limited to, melt recrystallization, melt cooling, solvent recrystallization, recrystallization in confined spaces such as, e.g., in nanopores or capillaries, recrystallization on surfaces or templates such as, e.g., on polymers, recrystallization in the presence of additives, such as, e.g., co-crystal counter-molecules, desolvation, dehydration, rapid evaporation, rapid cooling, slow cooling, vapor diffusion, sublimation, grinding and solvent-drop grinding.

Unless clearly indicated otherwise, “an individual” as used herein intends a mammal, including but not limited to a primate, human, bovine, horse, feline, canine, or rodent. In one variation, the individual is a human.

As used herein, “treatment” or “treating” is an approach for obtaining beneficial or desired results including clinical results. Beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. Also encompassed by “treatment” is a reduction of pathological consequence of fibrosis. The methods of the invention contemplate any one or more of these aspects of treatment.

As used herein, the term “effective amount” intends such amount of a compound or crystalline form of the invention which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents (e.g., a compound or crystalline form), and a single agent may be considered to be given in an effective amount if, in conjunction with one or more other agents, a desirable or beneficial result may be or is achieved. Suitable doses of any of the co-administered compounds or crystalline forms may optionally be lowered due to the combined action (e.g., additive or synergistic effects) of the compounds or crystalline forms.

As used herein, a “therapeutically effective amount” refers to an amount of a compound or crystalline form sufficient to produce a desired therapeutic outcome. It is understood that “therapeutically effective amount” and “effective amount” may be used interchangeably.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, the term “controlled release” refers to a drug-containing formulation or fraction thereof in which release of the drug is not immediate, i.e., with a “controlled release” formulation, administration does not result in immediate release of the drug into an absorption pool. The term encompasses depot formulations designed to gradually release the drug compound or crystalline form over an extended period of time. Controlled release formulations can include a wide variety of drug delivery systems, generally involving mixing the drug compound or crystalline form with carriers, polymers or other compounds having the desired release characteristics (e.g., pH-dependent or non-pH-dependent solubility, different degrees of water solubility, and the like) and formulating the mixture according to the desired route of delivery (e.g., coated capsules, implantable reservoirs, injectable solutions containing biodegradable capsules, and the like).

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc =“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

Unless otherwise stated, “substantially pure” intends a composition that contains no more than about 10% impurity, such as a composition comprising less than about 9%, about 7%, about 5%, about 3%, about 1%, or about 0.5% impurity.

As used herein, the term “substantially as shown in” when referring, for example, to an XRPD pattern, a DSC graph, a TGA graph, or a GVS graph, includes a pattern or graph that is not necessarily identical to those depicted herein, but that falls within the limits of experimental error or deviations when considered by one of ordinary skill in the art.

It is understood that aspects and embodiments described herein as “comprising” include “consisting of” and “consisting essentially of” embodiments.

### Crystalline Forms

### Form I

In some embodiments, provided herein is a crystalline form of Compound 1 (Form I).

In some embodiments, provided herein is a substantially anhydrous crystalline form of Compound 1 (e.g., containing less than about 2%, about 1%, about 0.5%, about 0.1%, or about 0.01% of water by weight) (Form I).

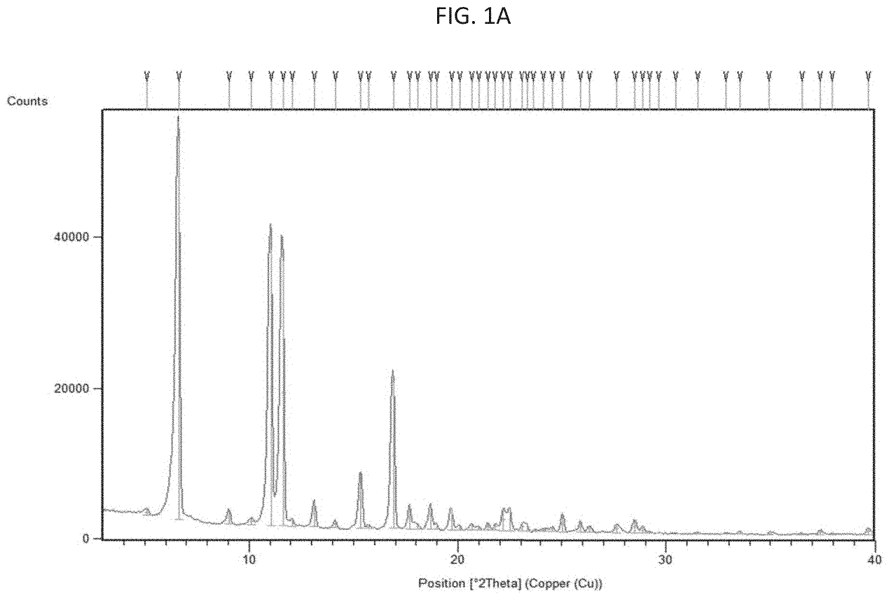

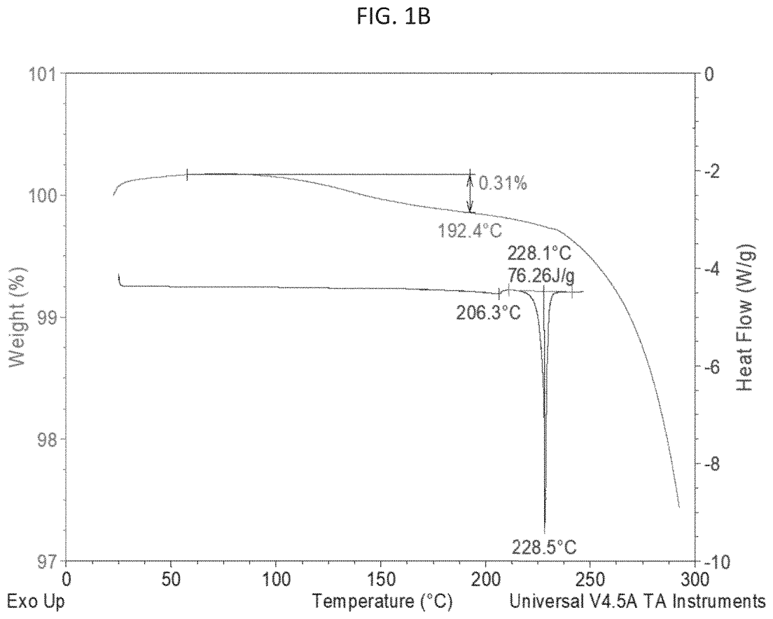

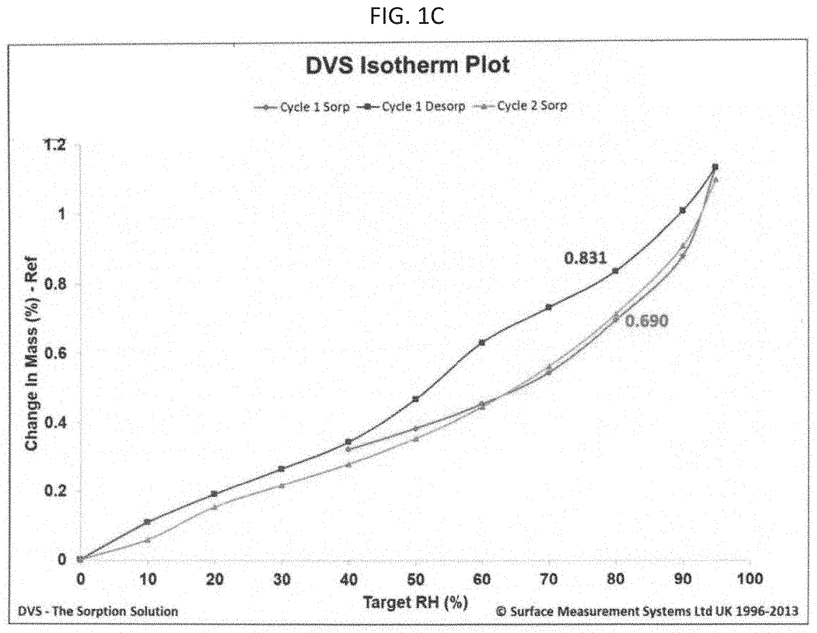

In some embodiments, Form I has an XRPD pattern substantially as shown in FIG. 1A. Positions of peaks and relative peak intensities that may be observed for the crystalline form using XRPD are shown in Table 1.

TABLE 1

| Pos. [°2θ] | Rel. Int. [%] |
|---|---|
| 5.1 | 1.69 |
| 6.6 | 100 |
| 9.1 | 3.71 |
| 10.1 | 1.94 |
| 11.1 | 72.9 |
| 11.6 | 70.13 |
| 12.1 | 2.08 |
| 13.1 | 6.75 |
| 14.1 | 1.88 |
| 15.3 | 14.76 |
| 15.7 | 1 |
| 16.9 | 38.95 |
| 17.7 | 6.38 |
| 18.1 | 1.18 |
| 18.7 | 6.61 |
| 19.0 | 1.41 |
| 19.7 | 5.53 |
| 20.1 | 1.15 |
| 20.7 | 1.62 |
| 21.0 | 0.89 |
| 21.4 | 1.77 |
| 21.8 | 1.77 |
| 22.2 | 5.61 |
| 22.5 | 5.87 |
| 23.1 | 1.99 |
| 23.3 | 1.78 |
| 23.7 | 0.39 |
| 24.1 | 0.84 |
| 24.6 | 1.22 |
| 25.1 | 4.84 |
| 25.9 | 2.95 |
| 26.4 | 1.67 |
| 27.6 | 2.27 |
| 28.5 | 3.46 |
| 28.9 | 1.73 |
| 29.2 | 0.47 |
| 29.7 | 0.28 |
| 30.5 | 0.13 |
| 31.5 | 0.39 |
| 32.9 | 0.38 |
| 33.5 | 0.89 |
| 35.0 | 0.66 |
| 36.5 | 0.4 |
| 37.4 | 1.22 |
| 37.9 | 0.26 |
| 39.7 | 1.83 |

In some embodiments, Form I has an XRPD pattern comprising peaks provided in Table 1. In some embodiments, Form I has an XRPD pattern comprising one or more (e.g., at least one, at least two, at least three, at least four, at least five, at least six, at least seven, at least eight, at least nine, or at least ten) of the peaks at angles 2-theta in the XRPD pattern substantially as shown in FIG. 1A, or as provided in Table 1. It should be understood that relative intensities and peak assignments can vary depending on a number of factors, including sample preparation, mounting, the instrument and analytical procedure and settings used to obtain the spectrum, temperature effects on the unit cell, and extent of solvation, e.g., hydration, of the sample. For example, relative peak intensities and peak assignments can vary within experimental error. In some embodiments, each peak assignment listed herein, including for Form I, can independently vary by ±0.4 degrees, ±0.3 degrees, ±0.2 degrees, or ±0.1 degrees 2-theta. In some embodiments, each peak assignment listed herein can independently vary by ±0.4 degrees 2-theta. In some embodiments, each peak assignment listed herein can independently vary by ±0.3 degrees 2-theta. In some embodiments, each peak assignment listed herein can independently vary by ±0.2 degrees 2-theta. In some embodiments, each peak assignment listed herein can independently vary by ±0.1 degrees 2-theta. It is also understood that an XRPD pattern substantially as shown in FIG. 1A encompasses an XRPD pattern in which the peak intensities of the one or more peaks differ from those of the corresponding peaks in FIG. 1A.

In some embodiments, Form I has an XRPD pattern comprising one or more peaks as assigned at angles 2-theta in degrees as recited in Table 1, each peak of which can independently vary in assignment at angle 2-theta in degrees as described herein. For example, Form I may have an XRPD pattern comprising peaks each assigned at an angle 2-theta in degrees of about 6.6 (e.g. 6.6±0.2), about 11.1 (e.g. 11.1±0.2), about 11.6 (e.g. 11.6±0.2), about 13.1 (e.g. 13.1±0.2), about 15.3 (e.g. 15.3±0.2), about 16.9 (e.g. 16.9±0.2), about 17.7 (e.g. 17.7±0.2), about 18.7 (e.g. 18.7±0.2), about 22.2 (e.g. 22.2±0.2), and/or about 22.5 (e.g. 22.5±0.2). In some embodiments, Form I has an XRPD pattern comprising one or more (e.g., at least one, at least two, at least three, at least four, at least five, at least six, at least seven, at least eight, at least nine, or at least ten) peaks each assigned at angles 2-theta in degrees of about 6.6 (e.g. 6.6±0.2), about 11.1 (e.g. 11.1±0.2), about 11.6 (e.g. 11.6±0.2), about 13.1 (e.g. 13.1±0.2), about 15.3 (e.g. 15.3±0.2), about 16.9 (e.g. 16.9±0.2), about 17.7 (e.g. 17.7±0.2), about 18.7 (e.g. 18.7±0.2), about 22.2 (e.g. 22.2±0.2), and/or about 22.5 (e.g. 22.5±0.2). In some embodiments, Form I has an XRPD pattern comprising peaks each assigned at angles 2-theta in degrees of about 6.6 (e.g. 6.6±0.2), about 11.1 (e.g. 11.1±0.2), about 11.6 (e.g. 11.6±0.2), about 15.3 (e.g. 15.3±0.2), and/or about 16.9 (e.g. 16.9±0.2). In some embodiments, Form I has an XRPD pattern comprising peaks each assigned at angles 2-theta in degrees of about 6.6 (e.g. 6.6±0.2), about 11.1 (e.g. 11.1±0.2), about 11.6 (e.g. 11.6±0.2), and/or about 16.9 (e.g. 16.9±0.2). In some embodiments, Form I has an XRPD pattern comprising peaks each assigned at angles 2-theta in degrees of about 6.6 (e.g. 6.6±0.2), about 11.1 (e.g. 11.1±0.2), and/or about 11.6 (e.g. 11.6±0.2). In some embodiments, Form I has an XRPD pattern comprising peaks each assigned at angles 2-theta in degrees of about 6.6 (e.g. 6.6±0.2) and/or about 11.6 (e.g. 11.6±0.2). In some embodiments, Form I has an XRPD pattern comprising peaks each assigned at angles 2-theta in degrees of about 6.6 (e.g. 6.6±0.2) and/or about 11.1 (e.g. 11.1±0.2). In some embodiments, Form I has an XRPD pattern comprising a peak assigned at angles 2-theta in degrees of about 6.6 (e.g. 6.6±0.2).

In some embodiments, Form I has a DSC graph substantially as shown in FIG. 1B. In some embodiments, Form I is characterized as having endotherm peaks at about 206.3° C. (e.g. 206.3±5° C., 206.3±4° C., 206.3±3° C., 206.3±2° C., 206.3±1° C., or 206.3±0.5° C.) and/or about 228.5° C. (e.g. 228.5±5° C., 228.5±4° C., 228.5±3° C., 228.5±2° C., 228.5±1° C., or 228.5±0.5° C.), as determined by DSC.

In some embodiments, Form I has a TGA graph substantially as shown in FIG. 1B. In some embodiments, Form I is characterized as showing a weight loss of about 0.31% (e.g., 0.31±0.10%, 0.31±0.09%, 0.31±0.08%, 0.31±0.07%, 0.31±0.06%, 0.31±0.05%, 0.31±0.04%, 0.31±0.03%, 0.31±0.02%, or 0.31±0.01%) after heating from room temperature to about 192.4° C. (e.g. 192.4±5° C., 192.4±4° C., 192.4±3° C., 192.4±2° C., 192.4±1° C., or 192.4±0.5° C.), as determined by TGA.

In some embodiments, Form I has a DVS graph substantially as shown in FIG. 1C.

In some embodiments of Form I, at least one, at least two, at least three, at least four, at least five, at least six, or all of the following (a)-(g) apply:

(a) Form I has an XRPD pattern comprising
(i) a peak assigned at angles 2-theta in degrees of about 6.6 (e.g. 6.6±0.2),
(ii) peaks each assigned at angles 2-theta in degrees of about 6.6 (e.g. 6.6±0.2) and/or about 11.6 (e.g. 11.6±0.2),
(iii) peaks at angles 2-theta of about 6.6 (e.g. 6.6±0.2), about 11.1 (e.g. 11.1±0.2), and/or about 11.6 (e.g. 11.6±0.2) degrees,
(iv) peaks each assigned at angles 2-theta in degrees of about 6.6 (e.g. 6.6±0.2), about 11.1 (e.g. 11.1±0.2), about 11.6 (e.g. 11.6±0.2), and/or about 16.9 (e.g. 16.9±0.2),
(v) peaks at angles 2-theta of about 6.6 (e.g. 6.6±0.2), about 11.1 (e.g. 11.1±0.2), about 11.6 (e.g. 11.6±0.2), about 15.3 (e.g. 15.3±0.2), and/or about 16.9 (e.g. 16.9±0.2) degrees, or
(vi) peaks at angles 2-theta of about 6.6 (e.g. 6.6±0.2), about 11.1 (e.g. 11.1±0.2), about 11.6 (e.g. 11.6±0.2), about 13.1 (e.g. 13.1±0.2), about 15.3 (e.g. 15.3±0.2), about 16.9 (e.g. 16.9±0.2), about 17.7 (e.g. 17.7±0.2), about 18.7 (e.g. 18.7±0.2), about 22.2 (e.g. 22.2±0.2), and/or about 22.5 (e.g. 22.5±0.2) degrees;
   (b) Form I has an XRPD pattern substantially as shown in FIG. 1A;
   (c) Form I is characterized as having endotherm peaks at about 206.3° C. (e.g. 206.3±5° C., 206.3±4° C., 206.3±3° C., 206.3±2° C., 206.3±1° C., or 206.3±0.5° C.) and/or about 228.5° C. (e.g. 228.5±5° C., 228.5±4° C., 228.5±3° C., 228.5±2° C., 228.5±1° C., or 228.5±0.5° C.), as determined by DSC;
   (d) Form I has a DSC graph substantially as shown in FIG. 1B;
   (e) Form I is characterized as showing a weight loss of about 0.31% (e.g., 0.31±0.10%, 0.31±0.09%, 0.31±0.08%, 0.31±0.07%, 0.31±0.06%, 0.31±0.05%, 0.31±0.04%, 0.31±0.03%, 0.31±0.02%, or 0.31±0.01%) after heating from room temperature to about 192.4° C. (e.g. 192.4±5° C., 192.4±4° C., 192.4±3° C., 192.4±2° C., 192.4±1° C., or 192.4±0.5° C.), as determined by TGA;
   (f) Form I has a TGA graph substantially as shown in FIG. 1B; and
   (g) Form I has a DVS graph substantially as shown in FIG. 1C.

### Form II

In some embodiments, provided herein is a crystalline form of Compound 1 (Form II).

In some embodiments, provided herein is a crystalline form of a chloroform solvate of Compound 1.

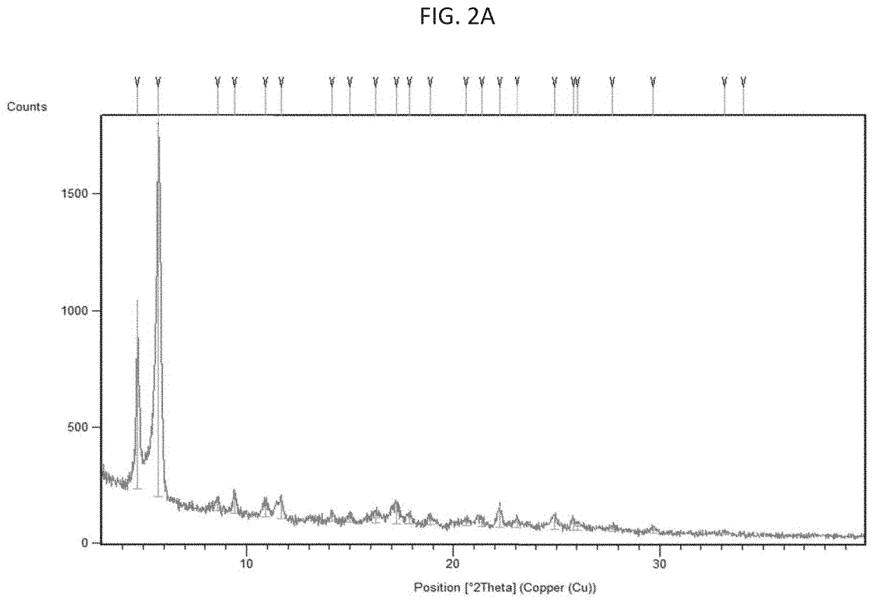

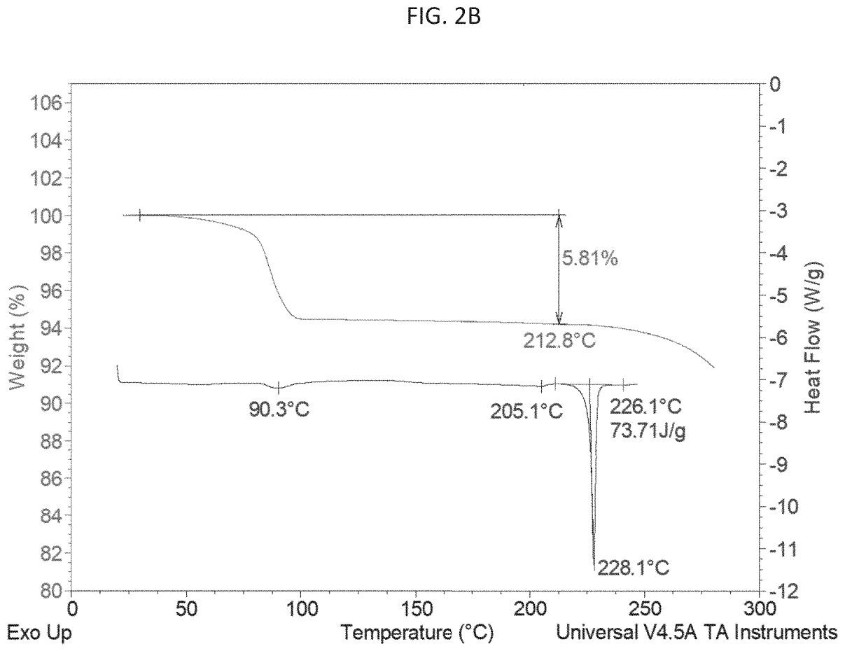

In some embodiments, Form II has an XRPD pattern substantially as shown in FIG. 2A. Positions of peaks and relative peak intensities that may be observed for the crystalline form using XRPD are shown in Table 2.

TABLE 2

| Pos. [°2θ] | Rel. Int. [%] |
|---|---|
| 4.7 | 50.75 |
| 5.7 | 100 |
| 8.6 | 3.03 |
| 9.4 | 6.24 |
| 10.9 | 4.46 |
| 11.7 | 5.81 |
| 14.1 | 2.45 |
| 15.0 | 1.9 |
| 16.3 | 2.81 |
| 17.2 | 5.83 |
| 17.9 | 2.92 |
| 18.9 | 2.33 |
| 20.6 | 1.88 |
| 21.4 | 2.1 |
| 22.2 | 5.76 |
| 23.1 | 2.76 |
| 24.9 | 3.96 |
| 25.8 | 3.13 |
| 26.0 | 2.04 |
| 27.7 | 1.31 |
| 29.7 | 1.4 |
| 33.1 | 0.8 |
| 34.1 | 0.73 |

In some embodiments, Form II has an XRPD pattern comprising peaks provided in Table 2. In some embodiments, Form II has an XRPD pattern comprising one or more (e.g., at least one, at least two, at least three, at least four, at least five, at least six, at least seven, at least eight, at least nine, or at least ten) of the peaks at angles 2-theta in the XRPD pattern substantially as shown in FIG. 2A, or as provided in Table 2. It should be understood that relative intensities and peak assignments can vary depending on a number of factors, including sample preparation, mounting, the instrument and analytical procedure and settings used to obtain the spectrum, temperature effects on the unit cell, and extent of solvation, e.g., hydration, of the sample. For example, relative peak intensities and peak assignments can vary within experimental error. In some embodiments, each peak assignment listed herein, including for Form II, can independently vary by ±0.4 degrees, ±0.3 degrees, ±0.2 degrees, or ±0.1 degrees 2-theta. In some embodiments, each peak assignment listed herein can independently vary by ±0.4 degrees 2-theta. In some embodiments, each peak assignment listed herein can independently vary by ±0.3 degrees 2-theta. In some embodiments, each peak assignment listed herein can independently vary by ±0.2 degrees 2-theta. In some embodiments, each peak assignment listed herein can independently vary by ±0.1 degrees 2-theta. It is also understood that an XRPD pattern substantially as shown in FIG. 2A encompasses an XRPD pattern in which the peak intensities of the one or more peaks differ from those of the corresponding peaks in FIG. 2A.

In some embodiments, Form II has an XRPD pattern comprising peaks as assigned at angles 2-theta in degrees as recited in Table 2, each peak of which can independently vary in assignment at angle 2-theta in degrees as described herein. For example, Form II may have an XRPD pattern comprising peaks each assigned at an angle 2-theta in degrees of about 4.7 (e.g. 4.7±0.2), about 5.7 (e.g. 5.7±0.2), about 8.6 (e.g. 8.6±0.2), about 9.4 (e.g. 9.4±0.2), about 10.9 (e.g. 10.9±0.2), about 11.7 (e.g. 11.7±0.2), about 17.2 (e.g. 17.2±0.2), about 22.2 (e.g. 22.2±0.2), about 24.9 (e.g. 24.9±0.2), and/or about 25.8 (e.g. 25.8±0.2). In some embodiments, Form II has an XRPD pattern comprising one or more (e.g., at least one, at least two, at least three, at least four, at least five, at least six, at least seven, at least eight, at least nine, or at least ten) peaks each assigned at angles 2-theta in degrees of about 4.7 (e.g. 4.7±0.2), about 5.7 (e.g. 5.7±0.2), about 8.6 (e.g. 8.6±0.2), about 9.4 (e.g. 9.4±0.2), about 10.9 (e.g. 10.9±0.2), about 11.7 (e.g. 11.7±0.2), about 17.2 (e.g. 17.2±0.2), about 22.2 (e.g. 22.2±0.2), about 24.9 (e.g. 24.9±0.2), and/or about 25.8 (e.g. 25.8±0.2). In some embodiments, Form II has an XRPD pattern comprising peaks each assigned at angles 2-theta in degrees of about 4.7 (e.g. 4.7±0.2), about 5.7 (e.g. 5.7±0.2), about 9.4 (e.g. 9.4±0.2), about 11.7 (e.g. 11.7±0.2), and/or about 17.2 (e.g. 17.2±0.2). In some embodiments, Form II has an XRPD pattern comprising peaks each assigned at angles 2-theta in degrees of about 4.7 (e.g. 4.7±0.2), about 5.7 (e.g. 5.7±0.2), and/or about 9.4 (e.g. 9.4±0.2). In some embodiments, Form II has an XRPD pattern comprising peaks each assigned at angles 2-theta in degrees of about 4.7 (e.g. 4.7±0.2) and/or about 5.7 (e.g. 5.7±0.2). In some embodiments, Form II has an XRPD pattern comprising a peaks assigned at angles 2-theta in degrees of about 5.7 (e.g. 5.7±0.2).

In some embodiments, Form II has a DSC graph substantially as shown in FIG. 2B. In some embodiments, Form II is characterized as having endotherm peaks at about 90.3° C. (e.g. 90.3±5° C., 90.3±4° C., 90.3±3° C., 90.3±2° C., 90.3±1° C., or 90.3±0.5° C.), about 205.1° C. (e.g. 205.1±5° C., 205.1±4° C., 205.1±3° C., 205.1±2° C., 205.1±1° C., or 205.1±0.5° C.), and/or about 228.1° C. (e.g. 228.1±5° C., 228.1±4° C., 228.1±3° C., 228.1±2° C., 228.1±1° C., or 228.1±0.5° C.), as determined by DSC.

In some embodiments, Form II has a TGA graph substantially as shown in FIG. 2B. In some embodiments, Form II is characterized as showing a weight loss of about 5.81% (e.g., 5.81±1%, 5.81±0.9%, 5.81±0.8%, 5.81±0.7%, 5.81±0.6%, 5.81±0.5%, 5.81±0.4%, 5.81±0.3%, 5.81±0.2%, or 5.81±0.1%) after heating from room temperature to about 212.8° C. (e.g. 212.8±5° C., 212.8±4° C., 212.8±3° C., 212.8±2° C., 212.8±1° C., or 212.8±0.5° C.), as determined by TGA.

In some embodiments of Form II, at least one, at least two, at least three, at least four, at least five, or all of the following (a)-(f) apply:

(a) Form II has an XRPD pattern comprising
(i) peaks at angles 2-theta of about 4.7 (e.g. 4.7±0.2) and/or about 5.7 (e.g. 5.7±0.2) degrees,
(ii) peaks at about 4.7 (e.g. 4.7±0.2), about 5.7 (e.g. 5.7±0.2), about 9.4 (e.g. 9.4±0.2), about 11.7 (e.g. 11.7±0.2), and/or about 17.2 (e.g. 17.2±0.2) degrees, or
(iii) peaks at about 4.7 (e.g. 4.7±0.2), about 5.7 (e.g. 5.7±0.2), about 8.6 (e.g. 8.6±0.2), about 9.4 (e.g. 9.4±0.2), about 10.9 (e.g. 10.9±0.2), about 11.7 (e.g. 11.7±0.2), about 17.2 (e.g. 17.2±0.2), about 22.2 (e.g. 22.2±0.2), about 24.9 (e.g. 24.9±0.2), and/or about 25.8 (e.g. 25.8±0.2) degrees;
   (b) Form II has an XRPD pattern substantially as shown in FIG. 2A;
   (c) Form II has a DSC graph substantially as shown in FIG. 2B;
   (d) Form II is characterized as having endotherm peaks at about 90.3° C. (e.g. 90.3±5° C., 90.3±4° C., 90.3±3° C., 90.3±2° C., 90.3±1° C., or 90.3±0.5° C.), about 205.1° C. (e.g. 205.1±5° C., 205.1±4° C., 205.1±3° C., 205.1±2° C., 205.1±1° C., or 205.1±0.5° C.), and/or about 228.1° C. (e.g. 228.1±5° C., 228.1±4° C., 228.1±3° C., 228.1±2° C., 228.1±1° C., or 228.1±0.5° C.), as determined by DSC;
   (e) Form II has a TGA graph substantially as shown in FIG. 2B; and
   (f) Form II is characterized as showing a weight loss of about 5.81% (e.g., 5.81±1%, 5.81±0.9%, 5.81±0.8%, 5.81±0.7%, 5.81±0.6%, 5.81±0.5%, 5.81±0.4%, 5.81±0.3%, 5.81±0.2%, or 5.81±0.1%) after heating from RT to about 212.8° C. (e.g. 212.8±5° C., 212.8±4° C., 212.8±3° C., 212.8±2° C., 212.8±1° C., or 212.8±0.5° C.), as determined by TGA.

### Form III

In some embodiments, provided herein is a crystalline form of Compound 1 (Form III).

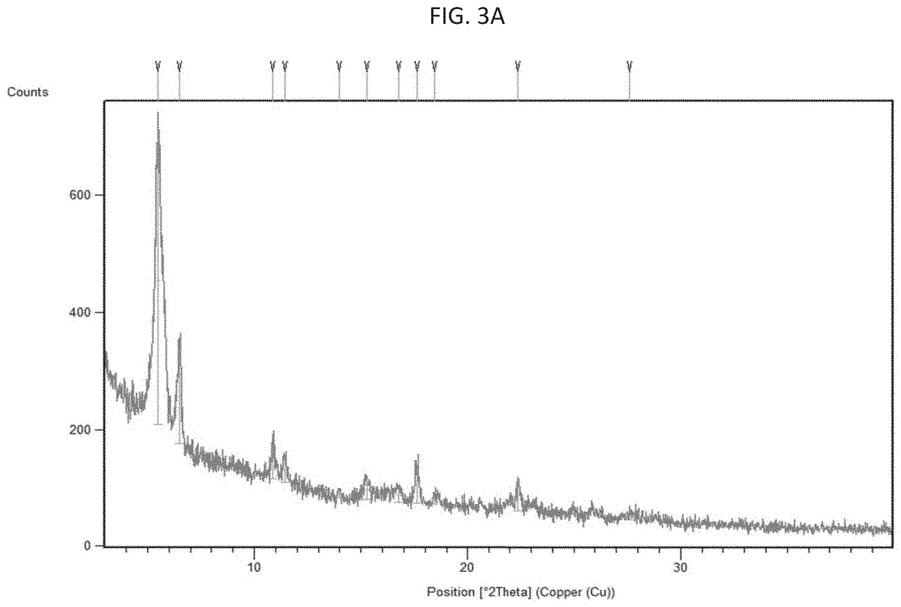

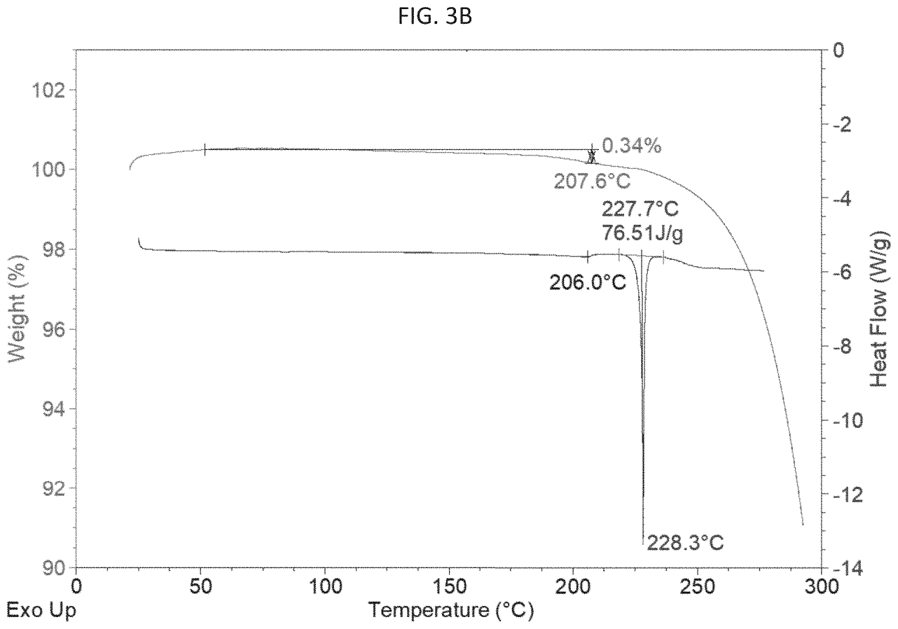

In some embodiments, Form III has an XRPD pattern substantially as shown in FIG. 3A. Positions of peaks and relative peak intensities that may be observed for the crystalline form using XRPD are shown in Table 3.

TABLE 3

| Pos. [°2θ] | Rel. Int. [%] |
|---|---|
| 5.5 | 100 |
| 6.5 | 33.36 |
| 10.9 | 12.27 |
| 11.5 | 9.05 |
| 14.0 | 2.77 |
| 15.3 | 7.09 |
| 16.8 | 4.81 |
| 17.6 | 12.48 |
| 18.5 | 3.47 |
| 22.4 | 10.73 |
| 27.6 | 1.9 |

In some embodiments, Form III has an XRPD pattern comprising peaks provided in Table 3. In some embodiments, Form III has an XRPD pattern comprising one or more (e.g., at least one, at least two, at least three, at least four, at least five, at least six, at least seven, at least eight, at least nine, or at least ten) of the peaks at angles 2-theta in the XRPD pattern substantially as shown in FIG. 3A, or as provided in Table 3. It should be understood that relative intensities and peak assignments can vary depending on a number of factors, including sample preparation, mounting, the instrument and analytical procedure and settings used to obtain the spectrum, temperature effects on the unit cell, and extent of solvation, e.g., hydration, of the sample. For example, relative peak intensities and peak assignments can vary within experimental error. In some embodiments, each peak assignment listed herein, including for Form III, can independently vary by ±0.4 degrees, ±0.3 degrees, ±0.2 degrees, or ±0.1 degrees 2-theta. In some embodiments, each peak assignment listed herein can independently vary by ±0.4 degrees 2-theta. In some embodiments, each peak assignment listed herein can independently vary by ±0.3 degrees 2-theta. In some embodiments, each peak assignment listed herein can independently vary by ±0.2 degrees 2-theta. In some embodiments, each peak assignment listed herein can independently vary by ±0.1 degrees 2-theta. It is also understood that an XRPD pattern substantially as shown in FIG. 3A encompasses an XRPD pattern in which the peak intensities of the one or more peaks differ from those of the corresponding peaks in FIG. 3A.

In some embodiments, Form III has an XRPD pattern comprising peaks as assigned at angles 2-theta in degrees as recited in Table 3, each peak of which can independently vary in assignment at angle 2-theta in degrees as described herein. For example, Form III may have an XRPD pattern comprising peaks each assigned at an angle 2-theta in degrees of about 5.5 (e.g. 5.5±0.2), about 6.5 (e.g. 6.5±0.2), about 10.9 (e.g. 10.9±0.2), about 11.5 (e.g. 11.5±0.2), about 14.0 (e.g. 14.0±0.2), about 15.3 (e.g. 15.3±0.2), about 16.8 (e.g. 16.8±0.2), about 17.6 (e.g. 17.6±0.2), about 18.5 (e.g. 18.5±0.2), and/or about 22.4 (e.g. 22.4±0.2). In some embodiments, Form III has an XRPD pattern comprising one or more (e.g., at least one, at least two, at least three, at least four, at least five, at least six, at least seven, at least eight, at least nine, or at least ten) peaks each assigned at angles 2-theta in degrees of about 5.5 (e.g. 5.5±0.2), about 6.5 (e.g. 6.5±0.2), about 10.9 (e.g. 10.9±0.2), about 11.5 (e.g. 11.5±0.2), about 14.0 (e.g. 14.0±0.2), about 15.3 (e.g. 15.3±0.2), about 16.8 (e.g. 16.8±0.2), about 17.6 (e.g. 17.6±0.2), about 18.5 (e.g. 18.5±0.2), and/or about 22.4 (e.g. 22.4±0.2). In some embodiments, Form III has an XRPD pattern comprising a peak assigned at angles 2-theta in degrees of about 5.5 (e.g. 5.5±0.2).

In some embodiments, Form III has a DSC graph substantially as shown in FIG. 3B. In some embodiments, Form III is characterized as having endotherm peaks about 206.0° C. (e.g. 206.0±5° C., 206.0±4° C., 206.0±3° C., 206.0±2° C., 206.0±1° C., or 206.0±0.5° C.), and/or about 228.3° C. (e.g. 228.3±5° C., 228.3±4° C., 228.3±3° C., 228.3±2° C., 228.3±1° C., or 228.3±0.5° C.), as characterized by DSC.

In some embodiments, Form III has a TGA graph substantially as shown in FIG. 3B. In some embodiments, Form III is characterized as showing a weight loss of about 0.34% (e.g., 0.34±0.10%, 0.34±0.09%, 0.34±0.08%, 0.34±0.07%, 0.34±0.06%, 0.34±0.05%, 0.34±0.04%, 0.34±0.03%, 0.34±0.02%, or 0.34±0.01%) after heating from room temperature to about 207.6° C. (e.g. 207.6±5° C., 207.6±4° C., 207.6±3° C., 207.6±2° C., 207.6±1° C., or 207.6±0.5° C.), as determined by TGA.

In some embodiments of Form III, at least one, at least two, at least three, at least four, at least five, or all of the following (a)-(f) apply:

(a) Form III has an XRPD pattern comprising a peak at angles 2-theta of about 5.5 (e.g. 5.5±0.2) degrees;
(b) Form III has an XRPD pattern substantially as shown in FIG. 3A;
(c) Form III has a DSC graph substantially as shown in FIG. 3B;
(d) Form III is characterized as having endotherm peaks at about 206.0 ° C. (e.g. 206.0±5 ° C., 206.0±4 ° C., 206.0±3 ° C., 206.0±2 ° C., 206.0±1 ° C., or 206.0±0.5 ° C.) and/or about 228.3° C. (e.g. 228.3±5 ° C., 228.3±4 ° C., 228.3±3 ° C., 228.3±2 ° C., 228.3±1 ° C., or 228.3±0.5 ° C.), as determined by DSC;
(e) Form III has a TGA graph substantially as shown in FIG. 3B; and
(f) Form III is characterized as showing a weight loss of about 0.34% (e.g., 0.34±0.10%, 0.34±0.09%, 0.34±0.08%, 0.34±0.07%, 0.34±0.06%, 0.34±0.05%, 0.34±0.04%, 0.34±0.03%, 0.34±0.02%, or 0.34±0.01%) after heating from RT to about 207.6° C. (e.g. 207.6±5° C., 207.6±4° C., 207.6±3° C., 207.6±2° C., 207.6±1° C., or 207.6±0.5° C.), as determined by TGA.

### Form IV

In some embodiments, provided herein is a crystalline form of Compound 1 (Form IV).

In some embodiments, provided herein is a crystalline form of a hydrate of Compound 1 (Form IV).

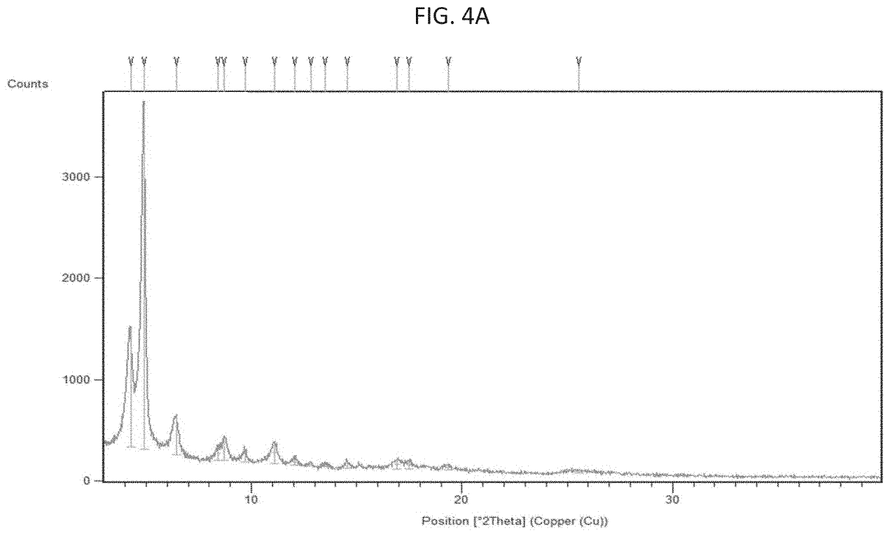

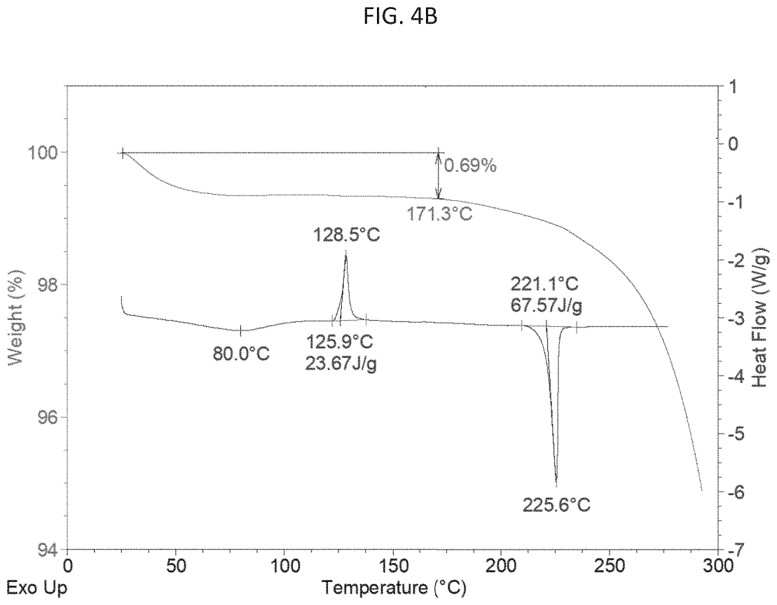

In some embodiments, Form IV has an XRPD pattern substantially as shown in FIG. 4A. Positions of peaks and relative peak intensities that may be observed for the crystalline form using XRPD are shown in Table 4.

TABLE 4

| Pos. [°2θ] | Rel. Int. [%] |
|---|---|
| 4.3 | 34.62 |
| 4.9 | 100 |
| 6.5 | 11.18 |
| 8.4 | 4.09 |
| 8.7 | 6.55 |
| 9.7 | 3.16 |
| 11.1 | 6.16 |
| 12.1 | 1.78 |
| 12.8 | 1.1 |
| 13.5 | 0.89 |
| 14.5 | 2.1 |
| 16.9 | 2.69 |
| 17.5 | 2.44 |
| 19.4 | 1.45 |
| 25.5 | 0.83 |

In some embodiments, Form IV has an XRPD pattern comprising peaks provided in Table 4. In some embodiments, Form IV has an XRPD pattern comprising one or more (e.g., at least one, at least two, at least three, at least four, at least five, at least six, at least seven, at least eight, at least nine, or at least ten) of the peaks at angles 2-theta in the XRPD pattern substantially as shown in FIG. 4A, or as provided in Table 4. It should be understood that relative intensities and peak assignments can vary depending on a number of factors, including sample preparation, mounting, the instrument and analytical procedure and settings used to obtain the spectrum, temperature effects on the unit cell, and extent of solvation, e.g., hydration, of the sample. For example, relative peak intensities and peak assignments can vary within experimental error. In some embodiments, each peak assignment listed herein, including for Form IV, can independently vary by ±0.4 degrees, ±0.3 degrees, ±0.2 degrees, or ±0.1 degrees 2-theta. In some embodiments, each peak assignment listed herein can independently vary by ±0.4 degrees 2-theta. In some embodiments, each peak assignment listed herein can independently vary by ±0.3 degrees 2-theta. In some embodiments, each peak assignment listed herein can independently vary by ±0.2 degrees 2-theta. In some embodiments, each peak assignment listed herein can independently vary by ±0.1 degrees 2-theta. It is also understood that an XRPD pattern substantially as shown in FIG. 4A encompasses an XRPD pattern in which the peak intensities of the one or more peaks differ from those of the corresponding peaks in FIG. 4A.

In some embodiments, Form IV has an XRPD pattern comprising peaks as assigned at angles 2-theta in degrees as recited in Table 4, each peak of which can independently vary in assignment at angle 2-theta in degrees as described herein. For example, Form IV may have an XRPD pattern comprising peaks each assigned at an angle 2-theta in degrees of about 4.3 (e.g. 4.3±0.2), about 4.9 (e.g. 4.9±0.2), about 6.5 (e.g. 6.5±0.2), about 8.4 (e.g. 8.4±0.2), about 8.7 (e.g. 8.7±0.2), about 9.7 (e.g. 9.7±0.2), about 11.1 (e.g. 11.1±0.2), about 14.5 (e.g. 14.5±0.2), about 16.9 (e.g. 16.9±0.2), and/or about 17.5 (e.g. 17.5±0.2). In some embodiments, Form IV has an XRPD pattern comprising one or more (e.g., at least one, at least two, at least three, at least four, at least five, at least six, at least seven, at least eight, at least nine, or at least ten) peaks each assigned at angles 2-theta in degrees of about 4.3 (e.g. 4.3±0.2), about 4.9 (e.g. 4.9±0.2), about 6.5 (e.g. 6.5±0.2), about 8.4 (e.g. 8.4±0.2), about 8.7 (e.g. 8.7±0.2), about 9.7 (e.g. 9.7±0.2), about 11.1 (e.g. 11.1±0.2), about 14.5 (e.g. 14.5±0.2), about 16.9 (e.g. 16.9±0.2), and/or about 17.5 (e.g. 17.5±0.2). In some embodiments, Form IV has an XRPD pattern comprising peaks each assigned at angles 2-theta in degrees of about 4.3 (e.g. 4.3±0.2), about 4.9 (e.g. 4.9±0.2), about 6.5 (e.g. 6.5±0.2), about 8.7 (e.g. 8.7±0.2), and/or about 11.1 (e.g. 11.1±0.2). In some embodiments, Form IV has an XRPD pattern comprising peaks each assigned at angles 2-theta in degrees of about 4.3 (e.g. 4.3±0.2), about 4.9 (e.g. 4.9±0.2), and/or about 6.5 (e.g. 6.5±0.2). In some embodiments, Form IV has an XRPD pattern comprising a peak at angles 2-theta in degrees of about 4.3 (e.g. 4.3±0.2). In some embodiments, Form IV has an XRPD pattern comprising a peak at angles 2-theta in degrees of about 4.9 (e.g. 4.9±0.2).

In some embodiments, Form IV has a DSC graph substantially as shown in FIG. 4B. In some embodiments, Form IV is characterized as having an endotherm peak at about 80.0° C. (e.g. 80.0±5° C., 80.0±4° C., 80.0±3° C., 80.0±2° C., 80.0±1° C., or 80.0±0.5° C.), an exotherm peak at about 128.5° C. (e.g. 128.5±5° C., 128.5±4° C., 128.5±3° C., 128.5±2° C., 128.5±1° C., or 128.5±0.5° C.), and/or an endotherm peak at about 225.6° C. (e.g. 225.6±5° C., 225.6±4° C., 225.6±3° C., 225.6±2° C., 225.6±1° C., or 225.6±0.5° C.), as determined by DSC.

In some embodiments, Form IV has a TGA graph substantially as shown in FIG. 4B. In some embodiments, Form IV is characterized as showing a weight loss of about 0.69% (e.g., 0.69±0.15%, 0.69±0.14%, 0.69±0.13%, 0.69±0.12%, 0.69±0.11%, 0.69±0.10%, 0.69±0.09%, 0.69±0.08%, 0.69±0.07%, 0.69±0.06%, 0.69±0.05%, 0.69±0.04%, 0.69±0.03%, 0.69±0.02%, or 0.69±0.1%) after heating from room temperature to about 171.3° C. (e.g. 171.3±5° C., 171.3±4° C., 171.3±3° C., 171.3±2° C., 171.3±1° C., or 171.3±0.5° C.), as determined by TGA.

In some embodiments of Form IV, at least one, at least two, at least three, at least four, at least five, or all of the following (a)-(g) apply:

(a) Form IV has an XRPD pattern comprising
(i) peaks at angles 2-theta of about 4.3 (e.g. 4.3±0.2), about 4.9 (e.g. 4.9±0.2), and/or about 6.5 (e.g. 6.5±0.2) degrees,
(ii) peaks at angles 2-theta of about 4.3 (e.g. 4.3±0.2), about 4.9 (e.g. 4.9±0.2), about 6.5 (e.g. 6.5±0.2), about 8.7 (e.g. 8.7±0.2), and/or about 11.1 (e.g. 11.1±0.2) degrees, or
(iii) peaks at angles 2-theta of about 4.3 (e.g. 4.3±0.2), about 4.9 (e.g. 4.9±0.2), about 6.5 (e.g. 6.5±0.2), about 8.4 (e.g. 8.4±0.2), about 8.7 (e.g. 8.7±0.2), about 9.7 (e.g. 9.7±0.2), about 11.1 (e.g. 11.1±0.2), about 14.5 (e.g. 14.5±0.2), about 16.9 (e.g. 16.9±0.2), and/or about 17.5 (e.g. 17.5±0.2) degrees;
   (b) Form IV has an XRPD pattern substantially as shown in FIG. 4A;
   (c) Form IV has a DSC graph substantially as shown in FIG. 4B;
   (d) Form IV is characterized as having an endotherm peak at about 80.0° C. (e.g. 80.0±5° C., 80.0±4° C., 80.0±3° C., 80.0±2° C., 80.0±1° C., or 80.0±0.5° C.), an exotherm peak at about 128.5° C. (e.g. 128.5±5° C., 128.5±4° C., 128.5±3° C., 128.5±2° C., 128.5±1° C., or 128.5±0.5° C.), and/or an endotherm peak at about 225.6° C. (e.g. 225.6±5° C., 225.6±4° C., 225.6±3° C., 225.6±2° C., 225.6±1° C., or 225.6±0.5° C.), as determined by DSC;
   (e) Form IV has a TGA graph substantially as shown in FIG. 4B; and
   (f) Form IV is characterized as showing a weight loss of about 0.69% (e.g., 0.69±0.15%, 0.69±0.14%, 0.69±0.13%, 0.69±0.12%, 0.69±0.11%, 0.69±0.10%, 0.69±0.09%, 0.69±0.08%, 0.69±0.07%, 0.69±0.06%, 0.69±0.05%, 0.69±0.04%, 0.69±0.03%, 0.69±0.02%, or 0.69±0.1%) after heating from room temperature to about 171.3° C. (e.g. 171.3±5° C., 171.3±4° C., 171.3±3° C., 171.3±2° C., 171.3±1° C., or 171.3±0.5° C.), as determined by TGA.

### Form V

In some embodiments, provided is a substantially anhydrous crystalline form of Compound 1 (e.g., containing less than about 1%, about 0.5%, about 0.1%, or about 0.01% of water by weight) (Form V).

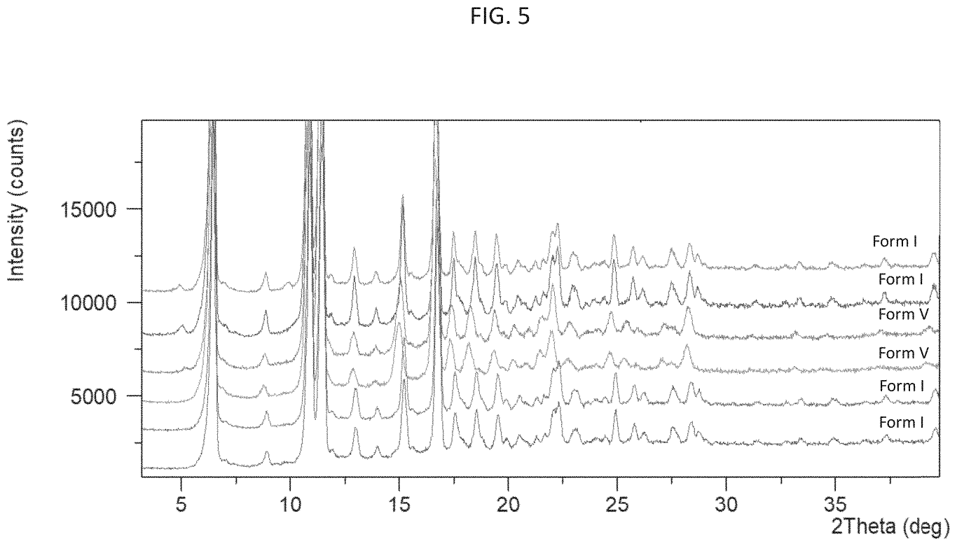

In some embodiments, Form V has an XRPD pattern substantially as shown in FIG. 5. Positions of peaks and relative peak intensities that may be observed for the crystalline form using XRPD are shown in Table 5.

TABLE 5

| Pos. [°2θ] | Rel. Int. [%] |
|---|---|
| 6.4 | 100 |
| 8.8 | 2.75 |
| 10.8 | 71.95 |
| 11.0 | 39.56 |
| 11.3 | 93.45 |
| 11.5 | 37.3 |
| 12.9 | 5.76 |
| 13.9 | 2.25 |
| 15.0 | 16.62 |
| 16.7 | 56.46 |
| 16.8 | 27.76 |
| 17.3 | 11.58 |
| 18.2 | 10.27 |
| 19.4 | 8.01 |
| 20.2 | 5.74 |
| 20.9 | 4.55 |
| 21.5 | 7.79 |
| 22.0 | 12.18 |
| 22.7 | 5.16 |
| 24.0 | 3.35 |
| 24.6 | 6.05 |
| 25.3 | 4.52 |
| 25.9 | 2.58 |
| 27.0 | 3.44 |
| 27.5 | 2.67 |
| 28.2 | 6.77 |
| 33.2 | 1.03 |
| 34.4 | 0.51 |
| 37.0 | 1.12 |
| 39.2 | 2 |

In some embodiments, Form V has an XRPD pattern comprising peaks provided in Table 5. In some embodiments, Form V has an XRPD pattern comprising one or more (e.g., at least one, at least two, at least three, at least four, at least five, at least six, at least seven, at least eight, at least nine, or at least ten) of the peaks at angles 2-theta with the greatest intensity in the XRPD pattern substantially as shown in FIG. 5, or as provided in Table 5. It should be understood that relative intensities and peak assignments can vary depending on a number of factors, including sample preparation, mounting, the instrument and analytical procedure and settings used to obtain the spectrum, temperature effects on the unit cell, and extent of solvation, e.g., hydration, of the sample. For example, relative peak intensities and peak assignments can vary within experimental error. In some embodiments, each peak assignment listed herein, including for Form V, can independently vary by ±0.4 degrees, ±0.3 degrees, ±0.2 degrees, or ±0.1 degrees 2-theta. In some embodiments, each peak assignment listed herein can independently vary by ±0.4 degrees 2-theta. In some embodiments, each peak assignment listed herein can independently vary by ±0.3 degrees 2-theta. In some embodiments, each peak assignment listed herein can independently vary by ±0.2 degrees 2-theta. In some embodiments, each peak assignment listed herein can independently vary by ±0.1 degrees 2-theta. It is also understood that an XRPD pattern substantially as shown in FIG. 5 encompasses an XRPD pattern in which the peak intensities of the one or more peaks differ from those of the corresponding peaks in FIG. 5.

### Salts

In some embodiments, provided is a salt form of Compound 1, such as a hydrochloride, a sulfate, a phosphate, an acetate, a maleate, a fumarate, a succinate, a malate, an adipate, a tartrate, a citrate, a nitrate, a tosylate, an oxalate, an ethanesulfonate, a benzenesulfonate, or a methanesulfonate salt. In some embodiments, the salt form is a crystalline form.

### Methods of Preparation

### Form I

Form I may be prepared according to the methods disclosed in Example 2. For example, in some embodiments, provided is a method of preparing Form I, comprising stirring a mixture of Compound 1 in a solvent, wherein the solvent comprises water, THF, dioxane, cyclohexane, EtOAc, n-heptane, DMF, iso-butanol, 2-MeTHF, cumene, toluene, EtOH, MEK, MIBK, n-BuOAc, DCM, ACN, MeOH, benzyl alcohol, 1-butanol, IPA, acetone, IPAc, 2-butanol, n-heptane, or a combination thereof. In some embodiments, the mixture is prepared as a slurry. In certain embodiments, the mixture is prepared as a slurry in a solvent comprising water, THF, dioxane, cyclohexane, EtOAc, n-heptane, DMF, iso-butanol, 2-MeTHF, cumene, toluene, EtOH, MEK, MIBK, n-BuOAc, a 1:1 mixture of DCM and ACN, a 1:1 mixture of DCM and MeOH, or a 1:1 mixture of benzyl alcohol and 1-butanol. In some embodiments, the stirring is conducted at room temperature. In some embodiments, the method is conducted at an elevated temperature such as at about 60° C.

### Form II

Form II may be prepared according to the methods disclosed in Example 2. For example, in some embodiments, provided is a method of preparing Form II, comprising slowly evaporating a mixture of Compound 1 in a solvent, wherein the solvent is chloroform.

### Form III

Form III may be prepared according to the methods disclosed in Example 2. For example, in some embodiments, provided is a method of preparing Form III, comprising adding an anti-solvent to a solution of Compound 1 in a solvent, wherein the solvent comprises chloroform and the anti-solvent comprises n-heptane.

### Form IV

Form IV may be prepared according to the methods disclosed in Example 2. For example, in some embodiments, provided is a method of preparing Form IV, comprising slowly cooling a solution of Compound 1 in a solvent, wherein the solvent comprises a mixture of DCM and MeOH (8:2 v/v).

### Form V

In some embodiments, provided is a method of preparing Form V, comprising heating Form I to a temperature of at least about 205° C.

### Pharmaceutical Compositions and Formulations

Pharmaceutical compositions of any of crystalline forms detailed herein are embraced by this invention. Thus, the invention includes pharmaceutical compositions comprising a crystalline form disclosed herein and a pharmaceutically acceptable carrier or excipient. In one embodiment, the pharmaceutical composition is a composition for controlled release of any of the crystalline forms detailed herein.

In some embodiments, provided is a composition comprising Form I. In some embodiments, the composition is substantially free of amorphous or non-crystalline form of Compound 1. In some embodiments of the composition comprising Form I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Form I. In some embodiments of the composition comprising Form I, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound 1 exists in Form I.

In some embodiments, provided is a composition comprising Form II. In some embodiments, the composition is substantially free of amorphous or non-crystalline form of Compound 1. In some embodiments of the composition comprising Form II, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Form II. In some embodiments of the composition comprising Form II, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound 1 exists in Form II.

In some embodiments, provided is a composition comprising Form III. In some embodiments, the composition is substantially free of amorphous or non-crystalline form of Compound 1. In some embodiments of the composition comprising Form III, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Form III. In some embodiments of the composition comprising Form III, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound 1 exists in Form III.

In some embodiments, provided is a composition comprising Form IV. In some embodiments, the composition is substantially free of amorphous or non-crystalline form of Compound 1. In some embodiments of the composition comprising Form IV, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Form IV. In some embodiments of the composition comprising Form IV, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound 1 exists in Form IV.

In some embodiments, provided is a composition comprising Form V. In some embodiments, the composition is substantially free of amorphous or non-crystalline form of Compound 1. In some embodiments of the composition comprising Form V, at least about 0.0001%, 0.001%, 0.01%, 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of the total composition is Form V. In some embodiments of the composition comprising Form V, at least about 0.1%, at least about 0.3%, at least about 0.5%, at least about 0.8%, at least about 1.0%, at least about 5.0%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99%, or at least 99.9% by weight of Compound 1 exists in Form V.

Crystalline forms or compositions disclosed herein may be formulated for any available delivery route, including an oral, mucosal (e.g., nasal, sublingual, vaginal, buccal or rectal), parenteral (e.g., intramuscular, subcutaneous or intravenous), topical or transdermal delivery form, or a form suitable for inhalation. A crystalline form or composition disclosed herein may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

Crystalline forms disclosed herein can be used in the preparation of a formulation, such as a pharmaceutical formulation, by combining the crystalline form as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21^{st} ed. (2005), which is incorporated herein by reference.

Crystalline forms disclosed herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

### Methods of Use

Crystalline forms and compositions detailed herein, such as a pharmaceutical composition containing a crystalline form of Compound 1 disclosed herein and a pharmaceutically acceptable carrier or excipient, may be used in methods of administration and treatment as provided herein. The crystalline forms and compositions may also be used in in vitro methods, such as in vitro methods of administering a crystalline form or composition to cells for screening purposes and/or for conducting quality control assays. In some embodiments of the methods detailed herein, the methods comprise administration of a crystalline form detailed herein as a monotherapy.

Provided herein is a method of treating a disease in an individual comprising administering an effective amount of a crystalline form disclosed herein to the individual. Further provided herein is a method of treating a proliferative disease in an individual, comprising administering an effective amount of the crystalline form to the individual. Also provided herein is a method of treating cancer in an individual comprising administering an effective amount of the crystalline form to the individual. In some embodiments, the crystalline form is administered to the individual according to a dosage and/or method of administration described herein.

In some embodiments, the cancer in the individual has one or more mutations or amplification or overexpression of the genes encoding cyclins or of the genes encoding the CDK or loss of endogenous INK4 inhibitors by gene deletion, mutation, or promoter hypermethylation, or other genetic events leading to overactivity of one or more of CDK1, CDK2, CDK4, CDK6 and CDK9. In some embodiments, the cancer in the individual has one or more mutations or amplification or overexpression of the genes encoding cyclins or of the genes encoding the CDK or loss of endogenous INK4 inhibitors by gene deletion, mutation, or promoter hypermethylation, or other genetic events leading to overactivity of CDK4/6 and one or more of CDK1, CDK2, and CDK9.

In some embodiments, provided is a method of treating a cancer in an individual, comprising (a) selecting the individual for treatment based on (i) the presence of phosphorylation of the retinoblastoma (Rb) protein in the cancer, or (ii) presence of mutations or amplification or overexpression of CDK4 or CDK6 in the cancer, and administering an effective amount of a crystalline form disclosed herein to the individual. In some embodiments, the cancer is assayed for the expression of phosphorylated Rb. In some embodiments, the cancer is assayed for the expression of CDK4 or CDK6. In some embodiments, the CDK4 or CDK6 gene of the cancer is sequenced to detect the one or more mutations or amplifications. In some embodiments, the CDK4 or CDK6 gene is sequenced by biopsying the cancer and sequencing the CDK4 or CDK6 gene from the biopsied cancer. In some embodiments, the CDK4 or CDK6 gene is sequenced by sequencing circulating-tumor DNA (ctDNA) from the individual. In some embodiments, the tumor is biopsied for upregulation of cyclin 2E wherein elevated levels of cyclin 2E can indicate resistance to CDK4/CDK6 inhibitor treatment.

In some embodiments, provided herein is use of a crystalline form disclosed herein in the manufacture of a medicament for treatment of a disease as disclosed herein. In some embodiments, provided herein is use of a crystalline form disclosed herein in the manufacture of a medicament for treatment of a proliferative disease, such as cancer.

In some embodiments, a crystalline form disclosed herein is used to treat an individual having a proliferative disease, such as cancer as described herein. In some embodiments, the individual is at risk of developing a proliferative disease, such as cancer. In some of these embodiments, the individual is determined to be at risk of developing cancer based upon one or more risk factors. In some of these embodiments, the risk factor is a family history and/or gene associated with cancer.

The present crystalline forms are believed to be effective for treating a variety of diseases and disorders. For example, in some embodiments, a crystalline form disclosed herein may be used to treat a proliferative disease, such as cancer. In some embodiments the cancer is a solid tumor. In some embodiments the cancer is any of adult and pediatric oncology, myxoid and round cell carcinoma, locally advanced tumors, metastatic cancer, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma, cutaneous T cell lymphoma, Hodgkin's lymphoma, non-Hodgkin's lymphoma, cancer of the adrenal cortex, ACTH-producing tumors, lung cancer, including small cell carcinoma and nonsmall cell cancers, breast cancer, including small cell carcinoma and ductal carcinoma, gastrointestinal cancers, including stomach cancer, colon cancer, colorectal cancer, polyps associated with colorectal neoplasia, pancreatic cancer, liver cancer, urological cancers, including bladder cancer, including primary superficial bladder tumors, invasive transitional cell carcinoma of the bladder, and muscle-invasive bladder cancer, prostate cancer, malignancies of the female genital tract, including ovarian carcinoma, primary peritoneal epithelial neoplasms, cervical carcinoma, uterine endometrial cancers, vaginal cancer, cancer of the vulva, uterine cancer and solid tumors in the ovarian follicle, malignancies of the male genital tract, including testicular cancer and penile cancer, kidney cancer, including renal cell carcinoma, brain cancer, including intrinsic brain tumors, neuroblastoma, astrocytic brain tumors, gliomas, metastatic tumor cell invasion in the central nervous system, bone cancers, including osteomas and osteosarcomas, skin cancers, including melanoma, tumor progression of human skin keratinocytes, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma.

In some embodiments, the cancer is defined by a molecular characteristic. In some embodiments, the cancer is an estrogen receptor-positive breast cancer. In some embodiments, the breast cancer is triple negative breast cancer. In some embodiments, the cancer is a KRAS-mutant non-small cell lung cancer. In some embodiments, the cancer is mantle cell lymphoma defined by a translocation involving CCND1 resulting in cyclin D1 overexpression.

In some embodiments, the crystalline forms and compositions described herein cause G₁-S cell cycle arrest in a cell (such as a cancer cell). In some embodiments, the cancer cell is a cancer cell from any of the cancer types described herein. In some embodiments, arrested cells enter a state of apoptosis. In some embodiments, arrested cells enter a state of senescence. In some embodiments, provided herein is a method of causing G₁-S checkpoint arrest in a cell comprising administering an effective amount of a crystalline form disclosed herein to the cell. In some embodiments, the G₁-S cell cycle arrest occurs in about 40% or more, about 50% or more, about 60% or more, about 70% or more, about 80% or more, about 85% or more, about 90% or more, about 95% or more, about 96% or more, about 97% or more, about 98% or more, or about 99% or more of cells in a cell population. In some embodiments, the G₁-S cell cycle arrest occurs in up to about 99%, up to about 98%, up to about 97%, up to about 96%, up to about 95%, up to about 90%, up to about 85%, or up to about 80% of cells in the cell population.

In some embodiments, provided herein is a method of inducing senescence in a cell comprising administering an effective amount of a crystalline form disclosed herein to the cell. In some embodiments, senescence is induced in about 40% or more, about 50% or more, about 60% or more, about 70% or more, about 80% or more, about 85% or more, about 90% or more, about 95% or more, about 96% or more, about 97% or more, about 98% or more, or about 99% or more of cells in a cell population. In some embodiments, senescence is induced in up to about 99%, up to about 98%, up to about 97%, up to about 96%, up to about 95%, up to about 90%, up to about 85%, or up to about 80% of cells in the cell population.

In some embodiments, provided herein is a method of inducing apoptosis in a cell comprising administering an effective amount of a crystalline form disclosed herein to the cell. In some embodiments, apoptosis is induced in about 40% or more, about 50% or more, about 60% or more, about 70% or more, about 80% or more, about 85% or more, about 90% or more, about 95% or more, about 96% or more, about 97% or more, about 98% or more, or about 99% or more of cells in a cell population. In some embodiments, apoptosis is induced in up to about 99%, up to about 98%, up to about 97%, up to about 96%, up to about 95%, up to about 90%, up to about 85%, or up to about 80% of cells in the cell population.

In some embodiments, provided herein is a method of inhibiting CDK4 or CDK6 in a cell comprising administering an effective amount of a crystalline form disclosed herein to the cell. In some embodiments, CDK4 or CDK6 is inhibited by about 10% or more, about 20% or more, about 30% or more, about 40% or more, about 50% or more, about 60% or more, about 70% or more, about 75% or more, about 80% or more, about 90% or more, about 95% or more, about 96% or more, about 97% or more, about 98% or more, or about 99% or more. In some embodiments, CDK4 or CDK6 is inhibited up to about 99%, up to about 98%, up to about 97%, up to about 96%, up to about 95%, up to about 90%, up to about 85%, up to about 80%, up to about 70%, or up to about 60%. In some embodiments, the activity of CDK4 or CDK6 is measured according to a kinase assay.

In some embodiments, provided herein is a method of inhibiting one or more of CDK1, CDK2, CDK4, CDK6, and CDK9 in a cell comprising administering an effective amount of a crystalline form disclosed herein to the cell. In some embodiments, one or more of CDK1, CDK2, CDK4, CDK6, and CDK9 is inhibited by about 10% or more, about 20% or more, about 30% or more, about 40% or more, about 50% or more, about 60% or more, about 70% or more, about 75% or more, about 80% or more, about 90% or more, about 95% or more, about 96% or more, about 97% or more, about 98% or more, or about 99% or more. In some embodiments, one or more of CDK1, CDK2, CDK4, CDK6, and CDK9 is inhibited up to about 99%, up to about 98%, up to about 97%, up to about 96%, up to about 95%, up to about 90%, up to about 85%, up to about 80%, up to about 70%, or up to about 60%. In some embodiments, the activity of one or more of CDK1, CDK2, CDK4, CDK6, and CDK9 is measured according to a kinase assay.

In some embodiments, provided herein is a method of inhibiting CDK4 or CDK6 comprising contacting CDK4 or CDK6 with an effective amount of Compound 1 derived from a crystalline form disclosed herein. In some embodiments, Compound 1 derived from the crystalline form binds to CDK4 or CDK6 with an IC₅₀ of less than 1 μM, less than 900 nM, less than 800 nM, less than 700 nM, less than 600 nM, less than 500 nM, less than 400 nM, less than 300 nM, less than 200 nM, less than 100 nM, less than 50 nM, less than 10 nM, less than 5 nM, less than 1 nM, or less than 0.5 nM. In some embodiments, Compound 1 derived from the crystalline form binds to CDK4 or CDK6 with an IC₅₀ between 0.1 nM and 1 nM, between 1 nM and 5 nM, between 5 nM and 10 nM, between 10 nM and 50 nM, between 50 nM and 100 nM, between 100 nM and 200 nM, between 200 nM and 300 nM, between 300 nM and 400 nM, between 400 nM and 500 nM, between 500 nM and 600 nM, between 600 nM and 700 nM, between 700 nM and 800 nM, between 800 nM and 900 nM, or between 900 nM and 1 μM. In some embodiments, the IC₅₀ is measured according to a kinase assay. In some embodiments, the IC₅₀ is measured according to a cell proliferation assay.

In some embodiments, provided herein is a method of inhibiting one or more of CDK1, CDK2, CDK4, CDK6, and CDK9 comprising contacting one or more of CDK1, CDK2, CDK4, CDK6, and CDK9 with an effective amount of Compound 1 derived from a crystalline form disclosed herein. In some embodiments, Compound 1 derived from a crystalline form disclosed herein binds to one or more of CDK1, CDK2, CDK4, CDK6, and CDK9 with an IC₅₀ of less than 1 μM, less than 900 nM, less than 800 nM, less than 700 nM, less than 600 nM, less than 500 nM, less than 400 nM, less than 300 nM, less than 200 nM, less than 100 nM, less than 50 nM, less than 10 nM, less than 5 nM, less than 1 nM, or less than 0.5 nM. In some embodiments, Compound 1 derived from a crystalline form disclosed herein binds to one or more of CDK1, CDK2, CDK4, CDK6, and CDK9 with an IC₅₀ between 0.1 nM and 1 nM, between 1 nM and 5 nM, between 5 nM and 10 nM, between 10 nM and 50 nM, between 50 nM and 100 nM, between 100 nM and 200 nM, between 200 nM and 300 nM, between 300 nM and 400 nM, between 400 nM and 500 nM, between 500 nM and 600 nM, between 600 nM and 700 nM, between 700 nM and 800 nM, between 800 nM and 900 nM, or between 900 nM and 1 μM. In some embodiments, the IC₅₀ is measured according to a kinase assay. In some embodiments, the IC₅₀ is measured according to a cell proliferation assay.

In some embodiments, provided herein is a method of modulating CDK4/6 in an individual, comprising administering to the individual a crystalline form disclosed herein. In some embodiments, provided herein is a method of modulating CDK4 and CDK 6 in an individual, comprising administering to the individual a crystalline form disclosed herein. In some embodiments, provided herein is a method of modulating CDK4/6 and one or more of CDK1, CDK2, and CDK9 in an individual, comprising administering to the individual a crystalline form disclosed herein. In some embodiments, provided herein is a method of modulating CDK4 and CDK 6 and one or more of CDK1, CDK2, and CDK9 in an individual, comprising administering to the individual a crystalline form disclosed herein. In some embodiments, Compound 1 derived from the crystalline form binds to one or more of CDK4/6 with an IC₅₀ of less than 1 μM, less than 900 nM, less than 800 nM, less than 700 nM, less than 600 nM, less than 500 nM, less than 400 nM, less than 300 nM, less than 200 nM, less than 100 nM, less than 50 nM, less than 10 nM, less than 5 nM, less than 1 nM, or less than 0.5 nM. In some embodiments, Compound 1 derived from the crystalline form binds to one or more of CDK4 and CDK6 with an IC₅₀ of less than 1 μM, less than 900 nM, less than 800 nM, less than 700 nM, less than 600 nM, less than 500 nM, less than 400 nM, less than 300 nM, less than 200 nM, less than 100 nM, less than 50 nM, less than 10 nM, less than 5 nM, less than 1 nM, or less than 0.5 nM. In some embodiments, Compound 1 derived from the crystalline form binds to one or more of CDK1, CDK2, CDK4, CDK6, and CDK9 with an IC₅₀ between 0.1 nM and 1 nM, between 1 nM and 5 nM, between 5 nM and 10 nM, between 10 nM and 50 nM, between 50 nM and 100 nM, between 100 nM and 200 nM, between 200 nM and 300 nM, between 300 nM and 400 nM, between 400 nM and 500 nM, between 500 nM and 600 nM, between 600 nM and 700 nM, between 700 nM and 800 nM, between 800 nM and 900 nM, or between 900 nM and 1 μM. In some embodiments, the IC₅₀ is measured according to a kinase assay. In some embodiments, the IC₅₀ is measured according to a cell proliferation assay.

In one embodiment, a crystalline form disclosed herein may enhance the antitumour immunity by increasing the functional capacity of tumour cells to present antigen or by reducing the immunosuppressive T_{Reg} population by suppressing their proliferation.

In some embodiments, provided herein is a method of inhibiting the proliferation of a cell, comprising contacting the cell with an effective amount of a crystalline form disclosed herein. In some embodiments, a crystalline form disclosed herein is effective in inhibiting the proliferation of the cell with an EC₅₀ of less than 5 μM, less than 2 μM, less than 1 μM, less than 900 nM, less than 800 nM, less than 700 nM, less than 600 nM, less than 500 nM, less than 400 nM, less than 300 nM, less than 200 nM, less than 100 nM, or less than 50 nM. In some embodiments, a crystalline form disclosed herein is effective in inhibiting the proliferation of the cell with an EC50 between 10 nM and 20 nM, between 20 nM and 50 nM, between 50 nM and 100 nM, between 100 nM and 500 nM, between 500 nM and 1 μM, between 1 μM and 2 μM, or between 2 μM and 5 μM. In some embodiments, the EC₅₀ is measured according to a cell proliferation assay.

### Combination Therapy

As provided herein, the presently disclosed crystalline forms may affect the immune system. Accordingly, the present crystalline forms may be used in combination with other anti-cancer agents or immunotherapies. In some embodiments, provided herein is a method of treating a disease in an individual comprising administering an effective amount of a crystalline form disclosed herein, and an additional therapeutic agent to the individual. In some embodiments, the second therapeutic agent is a cancer immunotherapy agent or an endocrine therapy agent or a chemotherapeutic agent. In some embodiments, the disease is a proliferative disease such as cancer.

In some embodiments, the additional therapeutic agent is a cancer immunotherapy agent. In some embodiments, the additional therapeutic agent is an immunostimulatory agent. In some embodiments, the additional therapeutic agent targets a checkpoint protein (for example an immune checkpoint inhibitor). In some embodiments, the additional therapeutic agent is effective to stimulate, enhance or improve an immune response against a tumor.

In another aspect provided herein is a combination therapy for the treatment of a disease, such as cancer. In some embodiments, provided is a method of treating a disease in an individual comprising administering an effective amount of a crystalline form disclosed herein, in combination with a radiation therapy.

In some embodiments, provided is a method of treating a disease in an individual comprising (a) administering an effective amount of a crystalline form disclosed herein, and (b) administering an effective amount of an endocrine therapy agent. In some embodiments, the endocrine therapy is antiestrogen therapy. In some embodiments, the endocrine therapy is an antihormone. In some embodiments, the antihormone is a selective estrogen receptor degrader (SERD, such as fulvestrant). In some embodiments, the antihormone is a selective estrogen receptor modulator. In some embodiments, the selective estrogen modulator is tamoxifen, toremifene or clomiphene. In some embodiments, the endocrine therapy is an aromatase inhibitor (such as letrozole). In some embodiments, the combination of a CDK4/6 inhibitor and endocrine therapy causes enhancement of G1-S cell-cycle arrest. In some embodiments, the antihormone is an antiandrogen. In some embodiments, the antiandrogen is an androgen biosynthesis inhibitor, such as abiraterone. In some embodiments, the antiandrogen is an androgen receptor antagonist such as enzalutamide, apalutamide or bicalutamide. In some embodiments, the combination of a CDK4/6 inhibitor and endocrine therapy causes enhanced entry into a senescent state. In some embodiments, the crystalline form is administered prior to, after, or simultaneously co-administered with the endocrine therapy agent. In some embodiments, the crystalline form is administered 1 or more hours (such as 2 or more hours, 4 or more hours, 8 or more hours, 12 or more hours, 24 or more hours, or 48 or more hours) prior to or after the endocrine therapy agent.

In some embodiments, provided is a method of treating a disease in an individual comprising (a) administering an effective amount of a crystalline form disclosed herein, and (b) administering an effective amount of a second chemotherapeutic agent. In some embodiments, the chemotherapeutic agent is another kinase inhibitor. In some embodiments, the crystalline form is administered prior to, after, or simultaneously co-administered with the second chemotherapeutic agent. In some embodiments, the crystalline form is administered 1 or more hours (such as 2 or more hours, 4 or more hours, 8 or more hours, 12 or more hours, 24 or more hours, or 48 or more hours) prior to or after the second chemotherapeutic agent.

Examples of chemotherapeutic agents that can be used in combination with a crystalline form disclosed herein include DNA-targeted agents, a DNA alkylating agent (such as cyclophosphamide, mechlorethamine, chlorambucil, melphalan, dacarbazine, or nitrosoureas), a topoisomerase inhibitor (such as a Topoisomerase I inhibitor (e.g., irinotecan or topotecan) or a Topoisomerase II inhibitor (e.g., etoposide or teniposide)), an anthracycline (such as daunorubicin, doxorubicin, epirubicin, idarubicin, mitoxantrone, or valrubicin), a histone deacetylase inhibitor (such as vorinostat or romidepsin), a bromodomain inhibitor, other epigenetic inhibitors, a taxane (such as paclitaxel or docetaxel), a kinase inhibitor (such as bortezomib, erlotinib, gefitinib, imatinib, vemurafenib, vismodegib, ibrutinib), an anti-angiogenic inhibitor, a nucleotide analog or precursor analog (such as azacitidine, azathioprine, capecitabine, cytarabine, doxifluridine, 5-fluorouracil, gemcitabine, hydroxyurea, mercaptopurine, methotrexate, or tioguanine), or a platinum-based chemotherapeutic agent (such as cisplatin, carboplatin, or oxaliplatin), pemetrexed, or a combination thereof. In some embodiments, provided is a method of treating a disease in an individual comprising (a) administering an effective amount of a crystalline form disclosed herein, and (b) administering an effective amount of a kinase inhibitor (such as bortezomib, erlotinib, gefitinib, imatinib, vemurafenib, vismodegib, or ibrutinib). In some embodiments, the crystalline form is administered prior to, after, or simultaneously co-administered with the kinase inhibitor. In some embodiments, the crystalline form is administered 1 or more hours (such as 2 or more hours, 4 or more hours, 8 or more hours, 12 or more hours, 24 or more hours, or 48 or more hours) prior to or after the kinase inhibitor.

In some embodiments, provided is a method of treating a disease in an individual comprising (a) administering an effective amount of a crystalline form disclosed herein, and (b) administering an effective amount of a DNA damaging agent. In some embodiments, the crystalline form is administered prior to, after, or simultaneously co-administered with the DNA damaging agent. In some embodiments, the crystalline form is administered 1 or more hours (such as 2 or more hours, 4 or more hours, 8 or more hours, 12 or more hours, 24 or more hours, or 48 or more hours) prior to or after the DNA damaging agent.

In some embodiments, provided is a method of treating a disease in an individual comprising (a) administering an effective amount of a crystalline form disclosed herein, and (b) administering an effective amount of a DNA alkylating agent (such as cyclophosphamide, mechlorethamine, chlorambucil, melphalan, dacarbazine, or nitrosoureas). In some embodiments, the crystalline form is administered prior to, after, or simultaneously co-administered with the DNA alkylating agent. In some embodiments, the crystalline form is administered 1 or more hours (such as 2 or more hours, 4 or more hours, 8 or more hours, 12 or more hours, 24 or more hours, or 48 or more hours) prior to or after the DNA alkylating agent.

In some embodiments, provided is a method of treating a disease in an individual comprising (a) administering an effective amount of a crystalline form disclosed herein, and (b) administering an effective amount of a topoisomerase inhibitor (such as a Topoisomerase I inhibitor (e.g., irinotecan or topotecan) or a Topoisomerase II inhibitor (e.g., etoposide or teniposide)). In some embodiments, the crystalline form is administered prior to, after, or simultaneously co-administered with the topoisomerase inhibitor. In some embodiments, the crystalline form is administered 1 or more hours (such as 2 or more hours, 4 or more hours, 8 or more hours, 12 or more hours, 24 or more hours, or 48 or more hours) prior to or after the topoisomerase inhibitor.

In some embodiments, provided is a method of treating a disease in an individual comprising (a) administering an effective amount of a crystalline form disclosed herein, and (b) administering an effective amount of an anthracycline (such as daunorubicin, doxorubicin, epirubicin, idarubicin, mitoxantrone, or valrubicin). In some embodiments, the crystalline form is administered prior to, after, or simultaneously co-administered with the anthracycline. In some embodiments, the crystalline form is administered 1 or more hours (such as 2 or more hours, 4 or more hours, 8 or more hours, 12 or more hours, 24 or more hours, or 48 or more hours) prior to or after the anthracycline.

In some embodiments, provided is a method of treating a disease in an individual comprising (a) administering an effective amount of a crystalline form disclosed herein, and (b) administering an effective amount of a histone deacetylase inhibitor (such as vorinostat or romidepsin). In some embodiments, the crystalline form is administered prior to, after, or simultaneously co-administered with the histone deacetylase inhibitor. In some embodiments, the crystalline form is administered 1 or more hours (such as 2 or more hours, 4 or more hours, 8 or more hours, 12 or more hours, 24 or more hours, or 48 or more hours) prior to or after the histone deacetylase inhibitor.

In some embodiments, provided is a method of treating a disease in an individual comprising (a) administering an effective amount of a crystalline form disclosed herein, and (b) administering an effective amount of a taxane (such as paclitaxel or docetaxel). In some embodiments, the crystalline form is administered prior to, after, or simultaneously co-administered with the taxane. In some embodiments, the crystalline form is administered 1 or more hours (such as 2 or more hours, 4 or more hours, 8 or more hours, 12 or more hours, 24 or more hours, or 48 or more hours) prior to or after the taxane.

In some embodiments, provided is a method of treating a disease in an individual comprising (a) administering an effective amount of a crystalline form disclosed herein, and (b) administering an effective amount of a nucleotide analog or precursor analog (such as azacitidine, azathioprine, capecitabine, cytarabine, doxifluridine, 5-fluorouracil, gemcitabine, hydroxyurea, mercaptopurine, methotrexate, or tioguanine). In some embodiments, the crystalline form is administered prior to, after, or simultaneously co-administered with the nucleotide analog or precursor analog. In some embodiments, the crystalline form is administered 1 or more hours (such as 2 or more hours, 4 or more hours, 8 or more hours, 12 or more hours, 24 or more hours, or 48 or more hours) prior to or after the nucleotide analog or precursor analog.

In some embodiments, provided is a method of treating a disease in an individual comprising (a) administering an effective amount of a crystalline form disclosed herein, and (b) administering an effective amount of a platinum-based chemotherapeutic agent (such as cisplatin, carboplatin, or oxaliplatin). In some embodiments, the crystalline form is administered prior to, after, or simultaneously co-administered with the platinum-based chemotherapeutic agent. In some embodiments, the crystalline form is administered 1 or more hours (such as 2 or more hours, 4 or more hours, 8 or more hours, 12 or more hours, 24 or more hours, or 48 or more hours) prior to or after the platinum-based chemotherapeutic agent.

In some embodiments, provided is a method of treating a disease in an individual comprising (a) administering an effective amount of a crystalline form disclosed herein, and (b) administering an effective amount of pemetrexed. In some embodiments, the crystalline form is administered prior to, after, or simultaneously co-administered with the pemetrexed. In some embodiments, the crystalline form is administered 1 or more hours (such as 2 or more hours, 4 or more hours, 8 or more hours, 12 or more hours, 24 or more hours, or 48 or more hours) prior to or after the pemetrexed.

In some embodiments, provided is a method of treating a disease in an individual comprising (a) administering an effective amount of a crystalline form disclosed herein, and (b) administering an effective amount of a Bruton's tyrosine kinase (BTK) inhibitor. In some embodiments, the crystalline form is administered prior to, after, or simultaneously co-administered with the BTK inhibitor. In some embodiments, the crystalline form is administered 1 or more hours (such as 2 or more hours, 4 or more hours, 8 or more hours, 12 or more hours, 24 or more hours, or 48 or more hours) prior to or after the BTK inhibitor.

In some embodiments, provided is a method of treating a disease in an individual comprising (a) administering an effective amount of a crystalline form disclosed herein, and (b) administering an effective amount of a PI3K or Akt inhibitor. In some embodiments, the crystalline form is administered prior to, after, or simultaneously co-administered with the PI3K or Akt inhibitor. In some embodiments, the crystalline form is administered 1 or more hours (such as 2 or more hours, 4 or more hours, 8 or more hours, 12 or more hours, 24 or more hours, or 48 or more hours) prior to or after the PI3K or Akt inhibitor.

In some embodiments, provided is a method of treating a disease in an individual comprising (a) administering an effective amount of a crystalline form disclosed herein, and (b) administering an effective amount of a DNA damage repair (DDR) pathway inhibitor. In some embodiments, the crystalline form is administered prior to, after, or simultaneously co-administered with the DDR pathway inhibitor. In some embodiments, the crystalline form is administered 1 or more hours (such as 2 or more hours, 4 or more hours, 8 or more hours, 12 or more hours, 24 or more hours, or 48 or more hours) prior to or after the DDR pathway inhibitor. Examples of inhibitors of the DDR pathway include poly(ADP-ribose) polymerase (PARP) inhibitors (such as olaparib, rucaparib, niraparib, or talazoparib), ataxia telangiectasia mutated (ATM) protein inhibitors, ataxia telangiectasia and Rad3-related (ATR) protein inhibitors, checkpoint kinase 1 (Chk1) inhibitors, or combinations thereof.

In some embodiments, provided is a method of treating a disease in an individual comprising (a) administering an effective amount of a crystalline form disclosed herein, and (b) administering an effective amount of a PARP inhibitor (such as olaparib, rucaparib, niraparib, or talazoparib). In some embodiments, the crystalline form is administered prior to, after, or simultaneously co-administered with the PARP inhibitor. In some embodiments, the crystalline form is administered 1 or more hours (such as 2 or more hours, 4 or more hours, 8 or more hours, 12 or more hours, 24 or more hours, or 48 or more hours) prior to or after the PARP inhibitor.

In some embodiments, provided is a method of treating a disease in an individual comprising (a) administering an effective amount of a crystalline form disclosed herein, and (b) administering an effective amount of an ATM protein inhibitor. In some embodiments, the crystalline form is administered prior to, after, or simultaneously co-administered with the ATM protein inhibitor. In some embodiments, the crystalline form is administered 1 or more hours (such as 2 or more hours, 4 or more hours, 8 or more hours, 12 or more hours, 24 or more hours, or 48 or more hours) prior to or after the ATM protein inhibitor.

In some embodiments, provided is a method of treating a disease in an individual comprising (a) administering an effective amount of a crystalline form disclosed herein, and (b) administering an effective amount of an ATR protein inhibitor. In some embodiments, the crystalline form is administered prior to, after, or simultaneously co-administered with the ATR protein inhibitor. In some embodiments, the crystalline form is administered 1 or more hours (such as 2 or more hours, 4 or more hours, 8 or more hours, 12 or more hours, 24 or more hours, or 48 or more hours) prior to or after the ATR protein inhibitor.

In some embodiments, provided is a method of treating a disease in an individual comprising (a) administering an effective amount of a crystalline form disclosed herein, and (b) administering an effective amount of an Chk1 inhibitor. In some embodiments, the crystalline form is administered prior to, after, or simultaneously co-administered with the Chk1 inhibitor. In some embodiments, the crystalline form is administered 1 or more hours (such as 2 or more hours, 4 or more hours, 8 or more hours, 12 or more hours, 24 or more hours, or 48 or more hours) prior to or after the Chk1 inhibitor.

In some embodiments, provided is a method of treating a disease in an individual comprising (a) administering an effective amount of a crystalline form disclosed herein, and (b) administering an effective amount of a further CDK4/6 inhibitor. In some embodiments, the crystalline form is administered prior to, after, or simultaneously co-administered with the further CDK4/6 inhibitor. In some embodiments, the crystalline form is administered 1 or more hours (such as 2 or more hours, 4 or more hours, 8 or more hours, 12 or more hours, 24 or more hours, or 48 or more hours) prior to or after the further CDK4/6 inhibitor.

In another aspect, provided herein is a combination therapy in which a crystalline form disclosed herein is coadministered (which may be separately or simultaneously) with one or more additional agents that are effective in stimulating immune responses to thereby further enhance, stimulate or upregulate immune responses in a subject. For example, provided is a method for stimulating an immune response in a subject comprising administering to the subject a crystalline form disclosed herein and one or more immunostimulatory antibodies, such as an anti-PD-1 antibody, an anti-PD-L1 antibody and/or an anti-CTLA-4 antibody, such that an immune response is stimulated in the subject, for example to inhibit tumor growth. In one embodiment, the subject is administered a crystalline form disclosed herein and an anti-PD-1 antibody. In another embodiment, the subject is administered a crystalline form disclosed herein and an anti-PD-L1 antibody. In yet another embodiment, the subject is administered a crystalline form disclosed herein and an anti-CTLA-4 antibody. In another embodiment, the immunostimulatory antibody (e.g., anti-PD-1, anti-PD-L1 and/or anti-CTLA-4 antibody) is a human antibody. Alternatively, the immunostimulatory antibody can be, for example, a chimeric or humanized antibody (e.g., prepared from a mouse anti-PD-1, anti-PD-L1 and/or anti-CTLA-4 antibody).

In one embodiment, the present disclosure provides a method for treating a proliferative disease (e.g., cancer), comprising administering a crystalline form disclosed herein and an anti-PD-1 antibody to a subject. In further embodiments, the crystalline form is administered at a subtherapeutic dose, the anti-PD-1 antibody is administered at a subtherapeutic dose, or both are administered at a subtherapeutic dose. In another embodiment, the present disclosure provides a method for altering an adverse event associated with treatment of a hyperproliferative disease with an immunostimulatory agent, comprising administering a crystalline form disclosed herein and a subtherapeutic dose of anti-PD-1 antibody to a subject. In certain embodiments, the subject is human. In certain embodiments, the anti-PD-1 antibody is a human sequence monoclonal antibody.

In one embodiment, the present invention provides a method for treating a hyperproliferative disease (e.g., cancer), comprising administering a crystalline form disclosed herein and an anti-PD-L1 antibody to a subject. In further embodiments, the crystalline form is administered at a subtherapeutic dose, the anti-PD-L1 antibody is administered at a subtherapeutic dose, or both are administered at a subtherapeutic dose. In another embodiment, the present invention provides a method for altering an adverse event associated with treatment of a hyperproliferative disease with an immunostimulatory agent, comprising administering a crystalline form disclosed herein and a subtherapeutic dose of anti-PD-L1 antibody to a subject. In certain embodiments, the subject is human. In certain embodiments, the anti-PD-L1 antibody is a human sequence monoclonal antibody.

In certain embodiments, the combination of therapeutic agents discussed herein can be administered concurrently as a single composition in a pharmaceutically acceptable carrier, or concurrently as separate compositions each in a pharmaceutically acceptable carrier. In another embodiment, the combination of therapeutic agents can be administered sequentially. For example, an anti-CTLA-4 antibody and a crystalline form disclosed herein can be administered sequentially, such as anti-CTLA-4 antibody being administered first and the crystalline form second, or the crystalline form being administered first and anti-CTLA-4 antibody second. Additionally or alternatively, an anti-PD-1 antibody and a crystalline form disclosed herein can be administered sequentially, such as anti-PD-1 antibody being administered first and the crystalline form second, or the crystalline form being administered first and anti-PD-1 antibody second. Additionally or alternatively, an anti-PD-L1 antibody and a crystalline form disclosed herein can be administered sequentially, such as anti-PD-L1 antibody being administered first and the crystalline form second, or the crystalline form being administered first and anti-PD-L1 antibody second.

Furthermore, if more than one dose of the combination therapy is administered sequentially, the order of the sequential administration can be reversed or kept in the same order at each time point of administration, sequential administrations can be combined with concurrent administrations, or any combination thereof.

Optionally, the combination comprising a crystalline form disclosed herein can be further combined with an immunogenic agent, such as cancerous cells, purified tumor antigens (including recombinant proteins, peptides, and carbohydrate molecules), cells, and cells transfected with genes encoding immune stimulating cytokines.

A crystalline form disclosed herein can also be further combined with standard cancer treatments. For example, the crystalline form can be effectively combined with chemotherapeutic regimens. In these instances, it is possible to reduce the dose of other chemotherapeutic reagent administered with the combination of the instant disclosure. Other combination therapies with a compound of the crystalline form include radiation, surgery, or hormone deprivation. Angiogenesis inhibitors can also be combined with the crystalline form. Inhibition of angiogenesis leads to tumor cell death, which can be a source of tumor antigen fed into host antigen presentation pathways.

In another example, a crystalline form disclosed herein can be used in conjunction with anti-neoplastic antibodies. By way of example and not wishing to be bound by theory, treatment with an anti-cancer antibody or an anti-cancer antibody conjugated to a toxin can lead to cancer cell death (e.g., tumor cells) which would potentiate an immune response mediated by CTLA-4, PD-1, PD-L1 or the crystalline form. In an exemplary embodiment, a treatment of a hyperproliferative disease (e.g., a cancer tumor) can include an anti-cancer antibody in combination with a crystalline form disclosed herein and anti-CTLA-4 and/or anti-PD-1 and/or anti-PD-L1 antibodies, concurrently or sequentially or any combination thereof, which can potentiate anti-tumor immune responses by the host. Other antibodies that can be used to activate host immune responsiveness can be further used in combination with a crystalline form disclosed herein.

In some embodiments, a crystalline form disclosed herein can be combined with an anti-CD73 therapy, such as an anti-CD73 antibody.

In yet further embodiments, a crystalline form disclosed herein is administered in combination with another CDK4 or CDK6 inhibitor or other CDK inhibitor, for example, a CDK2E selective inhibitor. In some embodiments, a CDK2E inhibitor with potency selectivity in a CDK enzymatic activity assay of at least 5×, 10×, 50×, 100× or >100× relative to CDK1.

### Kits

The present disclosure further provides kits for carrying out the methods of the invention, which comprises one or more crystalline forms described herein or a composition comprising a crystalline form described herein. The kits may employ any of the crystalline forms disclosed herein. The kits may be used for any one or more of the uses described herein, and, accordingly, may contain instructions for the treatment of cancer.

Kits generally comprise suitable packaging. The kits may comprise one or more containers comprising any crystalline form described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a crystalline form as disclosed herein and/or a second pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the crystalline forms and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The invention can be further understood by reference to the following examples, which are provided by way of illustration and are not meant to be limiting.

### EXAMPLES

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

The following abbreviations may be used herein:

| | |
|---|---|
| XRPD | X-Ray Powder Diffraction |
| DSC | Differential Scanning Calorimetry |
| TGA | Thermogravimetric Analysis |
| DVS | Dynamic Vapor Sorption |
| equiv. or eq. | Equivalents |
| RH | Relative humidity |
| RT | Room temperature |
| MEK | Methyl ethyl ketone |
| IPAc | Isopropyl acetate |
| MIBK | 4-Methyl-2-pentanone |
| EtOH | Ethanol |
| EtOAc | Ethyl acetate |
| DMSO | Dimethyl sulfoxide |
| DMF | Dimethylformamide |
| TBME or MTBE | tert-Butyl methyl ether |
| THF | Tetrahydrofuran |
| 2-MeTHF | 2-Methyltetrahydrofuran |
| H₂O | Water |
| DCM | Dichloromethane |
| MeOH | Methanol |
| ACN | Acetonitrile |
| IPA | Isopropyl alcohol |
| NPA | n-Propyl alcohol |
| n-BuOAc | n-Butyl acetate |
| API | Active Pharmaceutical Ingredient |
| HPLC | High performance liquid chromatography |

The crystalline forms were characterized by various analytical techniques, including XRPD, DSC, TGA, and DVS using the procedures described below.

### XRPD

XRPD was performed with a Panalytical X'Pert³ X-ray Powder XRPD on a Si zero-background holder. The 2θ position was calibrated against a Panalytical Si reference standard disc. The XRPD parameters used are listed in the table below.

Parameters for XRPD test

| Parameters | Reflection Mode |
|---|---|
| X-Ray wavelength | Cu, Kα; |
| | Kα1 (Å): 1.540598 |
| | Kα2 (Å): 1.544426 |
| | intensity ratio Kα2/Kα1: 0.50 |
| X-Ray tube setting | 45 kV, 40 mA |
| Divergence slit | Fixed ⅛° |
| Scan mode | Continuous |
| Scan range (2θ/°) | 3°~40° |
| Step size (2θ/°) | 0.0131° |
| Scan step time (s) | 18.87 |
| Test time (s) | About 4 min 15 s |

### TGA and DSC

TGA data were collected using a TA Discovery550 TGA from TA Instruments. DSC was performed using a TA Q2000 DSC from TA Instruments. Detailed parameters used are listed in the table below.

Parameters for TGA and DSC test

| Parameters | TGA | DSC |
|---|---|---|
| Method | Ramp | Ramp |
| Sample pan | Platinum, open | Aluminum, crimped |
| Temperature | RT- desired temperature | RT- desired temperature |
| Heating rate | 10° C./min | 10° C./min |
| Purge gas | N₂ | N₂ |

### DVS

DVS was measured via a SMS (Surface Measurement Systems) DVS Intrinsic. The relative humidity at 25° C. were calibrated against the deliquescence point of LiCl, Mg(NO₃)₂ and KCl. Parameters for DVS test are listed in the table below.

Parameters for DVS test

| Parameters | DVS | |
|---|---|---|
| Temperature | 25° | C. |
| Sample size | 10~20 | mg |
| Gas and flow rate | N₂, 200 mL/min | |
| dm/dt | 0.002%/min | |
| Min. dm/dtstabilityduration | 10 | min |
| Max. equilibrium time | 180 | min |

| | |
|---|---|
| RH range | 0%-95% RH-0% RH-95% RH |
| RH step size | 10% |

### Example 1. Preparation of Compound 1

Compound 1 was prepared as disclosed in U.S. Patent Publication No. US 2019/0248774 A1.

### Example 2. Polymorph Screening

Polymorph screening experiments were performed using different crystallization or solid transition methods. The methods utilized and the crystal types identified are summarized in the table below.

| Method | No. of Experiments | Crystal Type |
|---|---|---|
| Anti-solvent addition | 10 | Form I, Form III, gel |
| Slurry at 60° C. | 20 | Form I, gel |
| Slurry at RT | 20 | Form I, gel |
| Solid vapor diffusion | 10 | Form I, Form II |
| Liquid vapor diffusion | 10 | Form I, Form II |
| Slow evaporation | 2 | Form I, Form II |
| Temperature cycling | 20 | Form I, gel |
| Slow cooling | 8 | Form I, Form II, Form IV, |
| | | limited sample |

Slurry experiments were conducted at RT in different solvent systems. About 20 mg of Compound 1 was suspended in 0.15-0.3 mL of solvent in a 2 mL glass vial. After the suspension was stirred magnetically for one day at RT, the remaining solids were isolated for XRPD analysis. Results are summarized in the table below.

| Solvent | Solid Form |
|---|---|
| H₂O | Form I |
| THF | Form I |
| Dioxane | Form I |
| Cyclohexane | Form I |
| EtOAc | Form I |
| n-Heptane | Form I |
| DMF | Form I |
| iso-Butanol | Form I |
| 2-MeTHF | Form I |
| Cumene | Form I |
| Toluene | Form I |
| EtOH | Form I |
| MEK | Form I |
| MIBK | Form I |
| n-BuOAc | Form I |
| DCM/ACN (1:1) | Form I |
| DCM/MeOH (1:1) | Form I |
| Benzyl alcohol/1-Butanol (1:1) | Form I |
| Acetic acid/IPA (1:5) | Gel |
| Acetic acid/IPAc (1:5) | Gel |

Slurry experiments were conducted at 60° C. in different solvent systems. Approximately 20 mg of Compound 1 was suspended in 0.3 mL of solvent in a 2 mL glass vial. After the suspension was stirred magnetically for one day at 60° C., the remaining solids were isolated for XRPD analysis. Results are summarized in the table below.

| Solvent | Solid Form |
|---|---|
| MeOH | Form I |
| EtOH | Form I |
| IPA | Form I |
| 1-Butanol | Form I |
| IPAc | Form I |
| DMSO | Form I |
| EtOAc | Form I |
| ACN | Form I |
| MIBK | Form I |
| THF | Form I |
| 2-MeTHF | Form I |
| n-Heptane | Form I |
| H₂O | Form I |
| Toluene | Form I |
| MEK | Form I |
| Benzyl alcohol/MIBK (1:1) | Form I |
| Chloroform/Cyclohexane (1:1) | Form I |
| Chloroform/DMF (1:1) | Form I |
| Acetic acid/n-BuOAc (1:5) | Gel |
| Acetic acid/2-Butanol (1:5) | Gel |

Liquid vapor diffusion experiments were conducted. Approximately 15 mg of Compound 1 was dissolved in 0.3 to 0.4 mL solvent to obtain a clear solution in a 3 mL vial. This solution was placed into a 20 mL vial with 3 mL of volatile anti-solvent. The 20 mL vial was sealed with a cap and kept at RT, allowing enough time for organic vapor to interact with the solution. The precipitates were isolated for XRPD analysis. The results are summarized in the table below.

| Solvent | Anti-solvent | Solid Form |
|---|---|---|
| Chloroform | 2-MeTHF | Form I |
| Chloroform | EtOAc | Form I |
| Chloroform | MEK | Form I |
| DCM | H₂O | Form I |
| DCM | n-Heptane | Form I |
| Chloroform | MeOH | Form II |
| Chloroform | IPA | Form II |
| Chloroform | EtOH | Form II |
| Chloroform | Cyclohexane | Form II |
| DCM | Cyclohexane | Form I |

Solid vapor diffusion experiments were conducted. Approximately 15 mg of Compound 1 was weighed into a 3 mL vial, which was placed into a 20 mL vial with 3 mL of volatile solvent. The 20 mL vial was sealed with a cap and kept at RT for 7 days, allowing solvent vapor to interact with the sample. The solids were texted by XRPD. The results are summarized in the table below.

| Solvent | Solid Form |
|---|---|
| 2-Butanol | Form I |
| MTBE | Form I |
| IPA | Form I |
| Acetone | Form I |
| MeOH | Form I |
| EtOAc | Form I |
| DCM | Form I |
| EtOH | Form I |
| Cyclohexane | Form I |
| Chloroform | Form II |

Anti-solvent addition experiments were conducted. Approximately 20 mg of Compound 1 was dissolved in 0.45-1.35 mL solvent to obtain a clear solution. The solution was magnetically stirred followed by addition of 0.2 mL anti-solvent stepwise until precipitate appeared or the total amount of anti-solvent reached 15.0 mL. The precipitate was isolated for XRPD analysis. Results are summarized in the table below.

| Solvent | Anti-solvent | Solid Form |
|---|---|---|
| Chloroform | n-Heptane | Form III |
| DCM* | 2-MeTHF | Form I |
| Benzyl alcohol* | 2-Butanol | Form III |
| Acetic acid* | H₂O | n/a** |
| Chloroform | Cyclohexane | Form I |
| DCM* | THF | n/a** |
| Benzyl alcohol* | Toluene | n/a** |
| Acetic acid* | IPA | n/a** |
| Acetic acid* | EtOAc | Gel |
| Benzyl alcohol* | MIBK | n/a** |

*Solids were obtained via slow evaporation at RT.
**Not enough solids for characterization.

Slow evaporation experiments were performed. Approximately 15 mg of Compound 1 was dissolved in 0.3 mL of solvent in a 2 mL glass vial. The visually clear solutions were allowed to evaporate slowly at RT. The solids were isolated for XRPD analysis. The results are summarized in the table below.

| Solvent | Solid Form |
|---|---|
| DCM | Form I |
| Chloroform | Form II |

Temperature cycling experiments were conducted. Approximately 20 mg of Compound 1 was suspended in 0.3 mL of solvent in a 2 mL glass vial at RT. The suspension was then heated to 50° C. and equilibrated for 2 hours. The slurry was slowly cooled down to 5° C. at a rate of 0.1° C./min and then heated to 50° C. in one hour. The cycle was repeated once more, followed by cooling to 5° C. at a rate of 0.1° C./min. The samples were stored at 5° C. Solids were isolated and analyzed using XRPD. Results are summarized in the table below.

| Solvent | Solid Form |
|---|---|
| ACN | Form I |
| IPA | Form I |
| Toluene | Form I |
| 2-MeTHF | Form I |
| Acetone | Form I |
| MEK | Form I |
| DMF | Form I |
| n-BuOAc | Form I |
| Cumene | Form I |
| THF | Form I |
| IPAc | Form I |
| iso-Butanol | Form I |
| H₂O | Form I |
| 2-Butanol | Form I |
| n-Heptane | Form I |
| Benzyl alcohol/1-Butanol (1:1) | Form I |
| Benzyl alcohol/EtOH (1:1) | Form I |
| Acetic acid/IPA (1:5) | Gel |
| Chloroform/MIBK (1:1) | Form I |
| DCM/EtOAc (1:1) | Form I |

Slow cooling experiments were conducted. Approximately 20 mg of Compound 1 was dissolved in 0.15-0.2 mL solvent to obtain a clear solution. The suspension was then heated to 50° C. and equilibrated for 2 hours. The suspension was filtered using a PTFE membrane (pore size of 0.20 μm). Filtrates were slowly cooled to 5° C. at a rate of 0.1° C./min. Solids were isolated and analyzed using XRPD. Results are summarized in the table below.

| Solvent | Solid Form |
|---|---|
| Chloroform | Form II |
| DCM* | Form I |
| DCM/ACN (8:2)* | Form I |
| DCM/MeOH (8:2)* | Form IV*** |
| Benzyl alcohol/1-Butanol (8:2)* | n/a** |
| Acetic acid/IPA (1:5)* | Limited sample |
| Benzyl alcohol/MIBK (8:2)* | n/a** |
| Chloroform/Cyclohexane (8:2) | Form II |

*Solids were obtained via slow evaporation at RT.
**Not enough solids for characterization.
***Solids were fully dissolved after heating at 50° C.

### Example 3. Preparation of Form I

Form I was prepared according to Example 2. The product was analyzed by XRPD, DSC, TGA, and DVS. The XRPD pattern is shown in FIG. 1A. The TGA and DSC graphs are shown in FIG. 1B. The DVS graph is shown in FIG. 1C.

### Example 4. Preparation of Form II

Form II was prepared by slowly evaporating a solution of Compound 1 in chloroform, according to Example 2. The product was analyzed by XRPD, DSC, and TGA. The XRPD pattern is shown in FIG. 2A. The TGA and DSC graphs are shown in FIG. 2B. Based on XPRD measurement, Form II started converting to Form I upon heating.

### Example 5. Preparation of Form III

Form III was prepared by anti-solvent addition, with chloroform as the solvent and n-heptane as the anti-solvent, according to Example 2. The product was analyzed by XRPD, DSC, and TGA. The XRPD pattern is shown in FIG. 3A. The TGA and DSC graphs are shown in FIG. 3B. Based on XPRD measurement, Form III started converting to Form I upon heating.

### Example 6. Preparation of Form IV

Form IV was prepared slow cooling a solution of Compound 1 in DCM/MeOH (8:2, v:v), according to Example 2. The product was analyzed by XRPD, DSC, and TGA. The XRPD pattern is shown in FIG. 4A. The TGA and DSC graphs are shown in FIG. 4B. Based on XPRD measurement, Form IV started converting to Form I upon heating.

### Example 7. Preparation of Form V

Form V was prepared by heating a sample of Form I to 205° C. The product was analyzed by XRPD. Form V was converted to Form I after cooling to room temperature. A comparison between XRPD patterns of Form I and XRPD patterns of Form V is shown in FIG. 5.

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

1. A crystalline form of a compound of Formula (1):

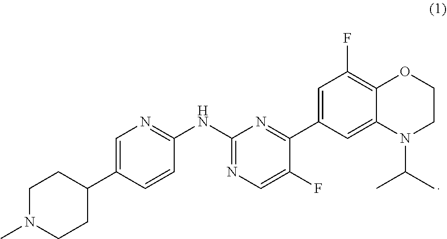 [SMILES: CC(C)N1CCOc2c(F)cc(-c3nc(Nc4ccc(C5CCN(C)CC5)cn4)ncc3F)cc21]

2. The crystalline form of claim 1, wherein the crystalline form is substantially anhydrous.

3. The crystalline form of claim 1, wherein the crystalline form is characterized as having an XRPD pattern comprising a peak at an angle 2-theta of about 6.6 degrees.

4. The crystalline form of claim 3, wherein the XRPD pattern further comprises a peak at an angle 2-theta of about 11.6 degrees.

5. The crystalline form of claim 4, wherein the XRPD pattern further comprises a peak at an angle 2-theta of about 11.1 degrees.

6. The crystalline form of claim 5, wherein the XRPD pattern further comprises a peak at an angle 2-theta of about 16.9 degrees.

7. The crystalline form of claim 6, wherein the XRPD pattern further comprises a peak at an angle 2-theta of about 15.3 degrees.

8. The crystalline form of claim 2, wherein the crystalline form is characterized as having an XRPD pattern substantially as shown in FIG. 1A.

9. The crystalline form of claim 2, wherein the crystalline form is characterized as having endotherm peaks at about 206.3° C. and/or about 228.5° C., as determined by DSC; or
is characterized as having a DSC graph substantially as shown in FIG. 1B.

10. (canceled)

11. The crystalline form of claim 2, wherein the crystalline form is characterized as showing a weight loss of about 0.31% after heating from room temperature to about 192.4° C., as determined by TGA; or
is characterized as having a TGA graph substantially as shown in FIG. 1B.

12. (canceled)

13. The crystalline form of claim 2, wherein the crystalline form is characterized as having a DVS graph substantially as shown in FIG. 1C.

14. The crystalline form of claim 1, wherein the crystalline form is a crystalline form of a chloroform solvate.

15. The crystalline form of claim 1, wherein the crystalline form is characterized as having an XRPD pattern comprising peaks at angles 2-theta of about 4.7 and about 5.7 degrees.

16. The crystalline form of claim 14, wherein the crystalline form is characterized as having an XRPD pattern substantially as shown in FIG. 2A.

17. The crystalline form of claim 14, wherein the crystalline form is characterized as having endotherm peaks at about 90.3° C., about 205.1° C., and/or about 228.1° C., as determined by DSC; or
is characterized as having a DSC graph substantially as shown in FIG. 2B.

18. (canceled)

19. The crystalline form of claim 14, wherein the crystalline form is characterized as showing a weight loss of about 5.81% after heating from room temperature to about 212.8° C., as determined by TGA; or
is characterized as having a TGA graph substantially as shown in FIG. 2B.

20. (canceled)

21. The crystalline form of claim 1, wherein the crystalline form is characterized as having an XRPD pattern comprising a peak at an angle 2-theta of about 5.5 degrees.

22. The crystalline form of claim 21, wherein the crystalline form is characterized as having an XRPD pattern substantially as shown in FIG. 3A.

23. The crystalline form of claim 21, wherein the crystalline form is characterized as having endotherm peaks at about 206.0° C. and/or about 228.3° C., as characterized by DSC; or
is characterized as having a DSC graph substantially as shown in FIG. 3B.

24. (canceled)

25. The crystalline form of claim 21, wherein the crystalline form is characterized as showing a weight loss of about 0.34% after heating from room temperature to about 207.6° C., as determined by TGA; or
is characterized as having a TGA graph substantially as shown in FIG. 3B.

26. (canceled)

27. The crystalline form of claim 1, wherein the crystalline form is a crystalline form of a hydrate.

28. The crystalline form of claim 27, wherein the crystalline form is characterized as having an XRPD pattern comprising peaks at angles 2-theta of about 4.3, about 4.9, and about 6.5 degrees.

29. The crystalline form of claim 27, wherein the crystalline form is characterized as having an XRPD pattern substantially as shown in FIG. 4A.

30. The crystalline form of claim 27, wherein the crystalline form is characterized as having an endotherm peak at about 80.0° C., an exotherm peak at about 128.5° C., and/or an endotherm peak at about 225.6° C., as determined by DSC; or
is characterized as having a DSC graph substantially as shown in FIG. 4B.

31. (canceled)

32. The crystalline form of claim 27, wherein the crystalline form is characterized as showing a weight loss of about 0.69% after heating from room temperature to about 171.3° C., as determined by TGA; or
is characterized as having a TGA graph substantially as shown in FIG. 4B.

33. (canceled)

34. A method of preparing the crystalline form of claim 2, comprising stirring a mixture of the compound in a solvent, wherein the solvent comprises water, THF, dioxane, cyclohexane, EtOAc, n-heptane, DMF, iso-butanol, 2-MeTHF, cumene, toluene, EtOH, MEK, MIBK, n-BuOAc, DCM, ACN, MeOH, benzyl alcohol, 1-butanol, IPA, acetone, IPAc, 2-butanol, n-heptane, or a combination thereof.

35. A method of preparing the crystalline form of claim 14, comprising slowly evaporating a mixture of the compound in a solvent, wherein the solvent is chloroform.

36. A method of preparing the crystalline form of claim 21, comprising adding an anti-solvent to a solution of the compound in a solvent, wherein the solvent comprises chloroform and the anti-solvent comprises n-heptane.

37. A method of preparing the crystalline form of claim 27, comprising slowly cooling a solution of the compound in a solvent, wherein the solvent comprises a mixture of DCM and MeOH (8:2 v/v).

38. A pharmaceutical composition comprising the crystalline form of claim 1, and a pharmaceutically acceptable carrier or excipient.

39. A kit comprising the crystalline form of claim 1.

40. A method of treating a cancer in an individual in need thereof comprising administering to the individual a therapeutically effective amount of the crystalline form of claim 1.

41. The method of claim 40, where the cancer is a breast cancer, brain cancer, colorectal cancer, lung cancer, gastric cancer, liver cancer, leukemia, lymphoma, mantle cell lymphoma, melanoma, ovarian cancer, pancreatic cancer, prostate cancer, adult hematopoietic or solid tumor, or pediatric tumor.

42-47. (canceled)

48. The method of claim 40, wherein the cancer comprises a mutated or overexpressed CDK gene.

49. (canceled)
